SEQUENCE LISTING (13 sequences)

SEQ ID NO: 1 - DNA, 951 bp, Homo sapiens
atggaatggagctgggtctttctcttcttcctgtcagtaactacaggtgtccactcccaggttcagctggttcagtccggggctgaggtgaagaagcctggggcctcagtgaaggtttcttgtcaggcttctggatacagattcagtaactttgttattcattgggtgcgccaggcccccggacagaggtttgagtggatgggatggatcaatccttacaacggaaacaaagaattttcagcgaagttccaggacagagtcacctttaccgcggacacatccgcgaacacagcctacatggagttgaggagcctcaggtctgcagacacggctgtttattattgtgcgagagtggggccatatagttgggatgattctccccaggacaattattatatggacgtctggggcaaagggaccacggtcatcgtgagctcagcttccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttggtgagaggccagcacagggagggagggtgtctgctggaagccaggctcagcgctcctgcctggacgcatcccggctatgcagccccagtccagggcagcaaggcaggccccgtctgcctcttcacccggaggcctctgcccgccccactcatgctcagggagagggtcttctggctttttccccaggctctgggcaggcacaggctaggtgcccctaa

SEQ ID NO: 2 - protein, 316 aa, Homo sapiens
MEWSWVFLFFLSVTTGVHSQVQLVQSGAEVKKPGASVKVSCQASGYRFSNFVIHWVRQAPGQRFEWMGWINPYNGNKEFSAKFQDRVTFTADTSANTAYMELRSLRSADTAVYYCARVGPYSWDDSPQDNYYMDVWGKGTTVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVGERPAQGGRVSAGSQAQRSCLDASRLCSPSPGQQGRPRLPLHPEASARPTHAQGEGLLAFSPGSGQAQARCP

SEQ ID NO: 3 - DNA, 708 bp, Homo sapiens
atgggtgtgcccactcaggtcctggggttgctgctgctgtggcttacagatgccagatgtgagatcgttctcacgcaggctccaggcaccctgtctctgtctccaggggaaagagccaccttctcctgtaggtccagtcacagcattcgcagccgccgcgtacgctggtaccagcacaaacctggccaggctccaaggctggtcatacatggtgtttccaatagggcctctggcatctcagacaggttcagcggcagtgggtctgggacagacttcactctcaccatcaccagagtggagcctgaagactttgcactgtactactgtcaggtctatggtgcctcctcgtacacttttggccaggggaccaaactggagaggaaacgaactgtgcctgcaccatctgtcttcatcttcccgccatctgatgagcagttgaaatctgggactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagatcgcccgtcacaaagagcttcaacaggggagagtgttaa

SEQ ID NO: 4 - protein, 235 aa, Homo sapiens
MGVPTQVLGLLLLWLTDARCEIVLTQAPGTLSLSPGERATFSCRSSHSIRSRRVRWYQHKPGQAPRLVIHGVSNRASGISDRFSGSGSGTDFTLTITRVEPEDFALYYCQVYGASSYTFGQGTKLERKRTVPAPSVFIFPPSDEQLKSGTASVVCLLNNFYPREAKVQWKVDNALQSGNSQESVTEQDSKDSTYSLSSTLTLSKADYEKHKVYACEVTHQGLRSPVTKSFNRGEC

SEQ ID NO: 5 - DNA, 26 bp, Artificial Sequence
  Description of Artificial Sequence Synthetic primer
gaccaaagtcatcgtctcctccgcct

SEQ ID NO: 6 - DNA, 26 bp, Artificial Sequence
  Description of Artificial Sequence Synthetic primer
cgccggtgccagtgtgctggaattcg

SEQ ID NO: 7 - DNA, 1854 bp, Artificial Sequence
  Description of Artificial Sequence Synthetic polynucleotide
atggaatggagctgggtctttctcttcttcctgtcagtaactacaggtgtccactcccaggttcagctggttcagtccggggctgaggtgaagaagcctggggcctcagtgaaggtttcttgtcaggcttctggatacagattcagtaactttgttattcattgggtgcgccaggcccccggacagaggtttgagtggatgggatggatcaatccttacaacggaaacaaagaattttcagcgaagttccaggacagagtcacctttaccgcggacacatccgcgaacacagcctacatggagttgaggagcctcaggtctgcagacacggctgtttattattgtgcgagagtggggccatatagttgggatgattctccccaggacaattattatatggacgtctggggcaaagggaccaaagtcatcgtctcctccgcctcggcctccccgtcgcccccccgcctcttcccgttggttctgtgctccccctccgactccgtctacaccgtcggctgcgccgccttcgacttccagccctcctccatcgccttcacgtggttcgattccaacaacagttccgtttccggtatggatgttatccctaaagtcatttccggtccaccttaccgggccgtcagtcgaatacagatgaatcaaagcgaagggaaagagaaacagcccttccggtgtcgggcggcgcatccacgcggcaacgtcgaggtcagcgtgatgaacccaggcccgattcccaccccgaatggcatcccccttttcgtcaccatgcaccccccgtcccgcgaggacttcgaaggccccttccgcaacgcctccatcctctgccagacccgcgggcgccgccgtcccaccgaggtcacgtggtacaaaaatggcagccccgtcgccgccgccgccaccaccgccaccaccgtcggccccgaagtggtggccgagagccgcatcagcgtcaccgaaagcgaatgggacaccggggccaccttcagctgcgtcgtggagggggagatgaggaacaccagcaagaggatggagtgcggattagaacccgtcgtgcagcaggacatcgccatccgcgtcatcacgccgtccttcgtggacatcttcatcagcaaatcggccacgctgacgtgccgggtgagcaacatggtgaacgccgacggcctggaggtgtcgtggtggaaggagaaggggggcaaactggagacggcgttggggaagagggtcctgcaaagcaacggcctctacacggtggacggggtggccacggtgtgcgccagcgaatgggacggaggggatggctacgtgtgtaaggtgaaccaccccgatctgctcttccccatggaggagaagatgaggaagacgaaagccagcaacgcccgccccccatccgtctacgtcttccccccccccacggaacaactgaacggcaaccaacggctcagcgtcacctgcatggctcagggcttcaaccccccccacctcttcgtcaggtggatgagaaacggggaacccctcccccaaagccaatcggtcacatcggcccccatggcggagaaccccgaaaatgagtcctacgtggcctacagcgttttgggggtgggggccgaagagtggggcgccggcaacgtctacacgtcgctggtgggccacgaagctctgcccctccagctggcccagaagtcggtggatagggcttcggatctcctccattggcctctggaggccgaagaggacgacgacatccaacgcctttgggccaccacctccaccttcatcgtcctcttcatcctcagcctcttctacagcgccgccgtcaccctcatcaaggtgaaatga

SEQ ID NO: 8 - protein, 617 aa, Artificial Sequence
  Description of Artificial Sequence Synthetic polypeptide
MEWSWVFLFFLSVTTGVHSQVQLVQSGAEVKKPGASVKVSCQASGYRFSNFVIHWVRQAPGQRFEWMGWINPYNGNKEFSAKFQDRVTFTADTSANTAYMELRSLRSADTAVYYCARVGPYSWDDSPQDNYYMDVWGKGTKVIVSSASASPSPPRLFPLVLCSPSDSVYTVGCAAFDFQPSSIAFTWFDSNNSSVSGMDVIPKVISGPPYRAVSRIQMNQSEGKEKQPFRCRAAHPRGNVEVSVMNPGPIPTPNGIPLFVTMHPPSREDFEGPFRNASILCQTRGRRRPTEVTWYKNGSPVAAAATTATTVGPEVVAESRISVTESEWDTGATFSCVVEGEMRNTSKRMECGLEPVVQQDIAIRVITPSFVDIFISKSATLTCRVSNMVNADGLEVSWWKEKGGKLETALGKRVLQSNGLYTVDGVATVCASEWDGGDGYVCKVNHPDLLFPMEEKMRKTKASNARPPSVYVFPPPTEQLNGNQRLSVTCMAQGFNPPHLFVRWMRNGEPLPQSQSVTSAPMAENPENESYVAYSVLGVGAEEWGAGNVYTSLVGHEALPLQLAQKSVDRASDLLHWPLEAEEDDDIQRLWATTSTFIVLFILSLFYSAAVTLIKVK

SEQ ID NO: 9 - DNA, 4648 bp, Human immunodeficiency virus
ctagagaaccatcagatgtttccagggtgccccaaggacctgaaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtattcccaataaagcctcttgctgtttgcatccgaatcgtggactcgctgatccttgggagggtctcctcagattgattgactgcccacctcgggggtctttcatttggaggttccaccgagatttggagacccctgcctagggaccaccgacccccccgccgggaggtaagctggccagcggtcgtttcgtgtctgtctctgtctttgtgcgtgtttgtgccggcatctaatgtttgcgcctgcgtctgtactagttagctaactagctctgtatctggcggacccgtggtggaactgacgagttctgaacacccggccgcaaccctgggagacgtcccagggactttgggggccgtttttgtggcccgacctgaggaagggagtcgatgtggaatccgaccccgtcaggatatgtggttctggtaggagacgagaacctaaaacagttcccgcctccgtctgaatttttgctttcggtttggaaccgaagccgcgcgtcttgtctgctgcagcgctgcagcatcgttctgtgttgtctctgtctgactgtgtttctgtatttgtctgaaaattagggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccgagatctgttaacctcgagatgataatatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccatcgataaaataaaagattttatttagtctccagaaaaaggggggaatgaaagaccccacctgtaggtttggcaaggaattgaggcctaactggccggtacctgagctcgctagcggggaatttccggggactttccgggaatttccggggactttccgggaatttccagatctggcctcggcggccaagcttagacactagagggtatataatggaagctcgacttccagcttggcaatccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtttatttagtctccagaaaaaggggggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtgtatccaataaaccctcttgcagttgcatccgacttgtggtctcgctgttccttgggagggtctcctctgagtgattgactacccgtcagcgggggtctttc

SEQ ID NO: 10 - protein, 674 aa, Human immunodeficiency virus
MRVKGIRKSYQYLWKGGTLLLGILMICSAVEKLWVTVYYGVPVWKEATTTLFCASDAKAYDTEVHNVWATHACVPTDPNPQEVVLENVTEHFNMWKNNMVEQMQEDIISLWDQSLKPCVKLTPLCVTLNCKDVNATNTTNDSEGTMERGEIKNCSFNITTSIRDEVQKEYALFYKLDVVPIDNNNTSYRLISCDTSVITQACPKISFEPIPIHYCAPAGFAILKCNDKTFNGKGPCKNVSTVQCTHGIRPVVSTQLLLNGSLAEEEVVIRSDNFTNNAKTIIVQLKESVEINCTRPNNNTRKSIHIGPGRAFYTTGEIIGDIRQAHCNISRAKWNDTLKQIVIKLREQFENKTIVFNHSSGGDPEIVMHSFNCGGEFFYCNSTQLFNSTWNNNTEGSNNTEGNTITLPCRIKQIINMWQEVGKAMYAPPIRGQIRCSSNITGLLLTRDGGINENGTEIFRPGGGDMRDNWRSELYKYKVVKIEPLGVAPTKAKRRVVQREKRAVGIGAVFLGFLGAAGSTMGAASMTLTVQARLLLSGIVQQQNNLLRAIEAQQRMLQLTVWGIKQLQARVLAVERYLGDQQLLGIWGCSGKLICTTAVPWNASWSNKSLDRIWNNMTWMEWEREIDNYTSEIYTLIEESQNQQEKNEQELLELDKWASLWNWFDITKWLWYIK

SEQ ID NO: 11 - protein, 670 aa, Human immunodeficiency virus
MRATEIRKNYQHLWKGGTLLLGMLMICSAAEQLWVTVYYGVPVWKEATTTLFCASDAKAYDTEVHNVWATHACVPTDPNPQEVKLENVTENFNMWKNNMVEQMHEDIISLWDQSLKPCVKLTPLCVTLNCTDLRNATNTTSSSWETMEKGEIKNCSFNITTSIRDKVQKEYALFYNLDVVPIDNASYRLISCNTSVITQACPKVSFEPIPIHYCAPAGFAILKCNDKKFNGTGPCTNVSTVQCTHGIRPVVSTQLLLNGSLAEEEIVIRSENFTNNAKTIIVQLNESVVINCTRPNNNTRKSINIGPGRALYTTGEIIGDIRQAHCNLSKTQWENTLEQIAIKLKEQFGNNKTIIFNPSSGGDPEIVTHSFNCGGEFFYCNSTQLFTWNDTRKLNNTGRNITLPCRIKQIINMWQEVGKAMYAPPIRGQIRCSSNITGLLLTRDGGKDTNGTEIFRPGGGDMRDNWRSELYKYKVVKIEPLGVAPTKAKRRVVQREKRAVGLGALFLGFLGAAGSTMGAASITLTVQARQLLSGIVQQQNNLLRAIEAQQHLLQLTVWGIKQLQARVLAVERYLRDQQLLGIWGCSGKLICTTTVPWNTSWSNKSLNEIWDNMTWMKWEREIDNYTHIIYSLIEQSQNQQEKNEQELLALDKWASLWNWFDITKWLWYIK

SEQ ID NO: 12 - protein, 673 aa, Human immunodeficiency virus
MPMGSLQPLATLYLLGMLVASVLAAGNLDLWVTVYYGVPVWKEAKTTLFCASDAKAYDKEVHNVWATHACVPTDPNPQEIVLENVTENFNMWKNDMVDQMHEDIISLWDQSLKPCVKLTPLCVTLNCKNVNISANANATATLNSSMNGEIKNCSFNTTTELRDKKQKVYALFYKPDVVPLNGGEHNETGEYILINCNSSTITQACPKVSFDPIPIHYCAPAGYAILKCNNKTFNGTGPCNNVSTVQCTHGIKPVVSTQLLLNGSLAEEEIIVRSENLTNNIKTIIVHLNKSVEIKCTRPNNNTRKSVRIGPGQTFYATGEIIGDIREAHCNISRETWNSTLIQVKEKLREHYNKTIKFEPSSGGDLEVTTHSFNCRGEFFYCDTTKLFNETKLFNESEYVDNKTIILPCRIKQIINMWQEVGRAMYAPPIEGNITCKSNITGLLLTWDGGENSTEGVFRPGGGNMKDNWRSELYKYKVVEIKPLGVAPTKSKRKVVGREKRAVGLGAVLLGFLGAAGSTMGAASITLTVQARQLLSGIVQQQSNLLRAIEAQQHLLQLTVWGIKQLQTRVLAIERYLKDQQLLGLWGCSGKLICATAVPWNSSWSNKSLGDIWDNMTWMQWDREISNYTNTIFRLLEDSQNQQEKNEKDLLALDSWKNLWNWFDITNWLWYIK

SEQ ID NO: 13 - protein, 6 aa, Unknown
  Description of Unknown Caspace-3 cleavage site peptide
DEVDGI

(19) United States
(12) Patent Application Publication
(10) Pub. No.: US 2013/0236905 A1
(43) Pub. Date: Sep. 12, 2013

(54) ASSAY FOR IDENTIFYING ANTIGENS THAT ACTIVATE B CELL RECEPTORS COMPRISING NEUTRALIZING ANTIBODIES

(71) Applicant: Christopher Marshall, Brooklyn, NY (US)

(72) Inventor: Christopher Marshall, Brooklyn, NY (US)

(21) Appl. No.: 13/261,520

(22) PCT Filed: May 17, 2011
(86) PCT No.: PCT/US2011/000883

§ 371 Date: Nov. 19, 2012

Related U.S. Application Data
(63) Continuation of application No. 12/800,636, filed on May 18, 2010, now abandoned.

(51) Int. Cl. G01N 33/50; C12Q 1/68

(52) U.S. Cl. CPC G01N 33/5052; C12Q 1/6897
USPC 435/6.13; 435/7.24

(57) ABSTRACT

The invention described herein provides a method for screening pathogenic viral envelope proteins and protein complexes to identify protein constructs with enhanced effectiveness as vaccine immunogens. The method is carried out by (i) expressing of a membrane-bound isotype of an antibody that has the same binding activity and specificity of an antibody that is known, or identified, to bind and neutralize the targeted virus, and that has the capacity to activate signaling pathways down-stream of B cell receptor ligand binding and activation—a modified neutralizing antibody-based B cell receptor; (ii) exposing the cell to antigen; and (iii) assay for signaling downstream of B cell receptor activation. The present invention also provides the antigens identified using the as say described herein, and neutralizing antibodies derived by immunization with the antigens identified using the assay described herein.

This application claims priority of U.S. Provisional Application No. 61/179,321 filed May 18^{th}. 2009, and U.S. application Ser. No. 12/800,636 filed May 18^{th}, 2010, both are which are incorporated-by-reference herein in their entirety.

### 1. FIELD OF THE INVENTION

The present invention relates to methods of identifying of viral antigens for commercial purposes (preventative and therapeutic vaccines), and to viral polypeptides and proteins so identified.

### 2. BACKGROUND OF THE INVENTION

The B-cell response to antigens, mediated through B cell receptors (BCR), is an essential component of the immune system. Immature B cells undergo a selection process based on antigen binding prior to leaving the bone marrow. Mature B cells recognize foreign antigens through B cell receptors and produce specific antibodies which bind the foreign antigens. To generate an efficient response to complex antigens, the BCR, BCR associated proteins, and T cell assistance are required. The antigen/receptor complex is internalized, and the antigen is proteolytically processed, and presented to T cells in the context of the major histocompatability complex molecules on the surface of the B cells; T cells activated by antigen presentation secrete a variety of lymphokines that induce B cell maturation.

### THE BRC SIGNALING COMPLEX

The B cell receptor is an immunoglobulin complex has the function of antigen binding and signaling when an antigen binds the receptor. It is present in the plasma membrane of B cells, and in its canonical form is a hetero-oligomeric structure composed of an antigen binding component, a disulfide bond complex consisting of two identical copies of a membrane-bound form of immunoglobulin heavy chains and two identical immunoglobulin light chains, and a signaling subunit, a heterodimer of the Ig-alpha and Ig-beta proteins (CD79a, and CD79b, respectively) non-covalently associated with the membrane-bound immunoglobulin heavy chains. Each B cell expresses one immunoglobulin but the population of B cells in each individual displays a wide variety of antigen specificity. When the B cell receptor binds to antigen, it initiates a signal through the cytoplasmic tails of Ig-alpha and Ig-beta chains that are each associated with distinct sets of downstream signaling/effector molecules.

Antigen binding of the BCR leads to activation of the Src family kinases Lyn, Blk and Fyn as well as the Syk and Btk tyrosine kinases, initiating complex signaling cascades involving multiple adaptor proteins, kinases, phosphatases, GTPases and transcription factors. The complexity of BCR signaling permits many distinct outcomes, including differentiation, survival, apoptosis, proliferation and tolerance. The outcome of the response is determined by the maturation state of the cell, the nature of the antigen, the magnitude and duration of BCR signaling, and signals from other receptors such as CD40. Many other transmembrane proteins, some of which are receptors, modulate specific elements of BCR signaling, including CD45, CD 19, CD22, PIR-B, and FcγRIIB1 (CD32). The magnitude and duration of BCR signaling are limited by negative feedback loops including those involving the Lyn/CD22/SHP-1 pathway, SHIP, Cbl, Dok-1, Dok-3, FcγRIIB1, PIR-B, and internalization of the BCR.

Early biochemical events in signal transduction, such as protein kinase activation and release of calcium ions, are similar for the two receptors (IgM and IgD); their subsequent biological effects, however, are different. Antigen binding or cross-linking of the IgM receptor leads to apoptosis, while binding of IgM and IgD, or IgD alone, does not. Binding to IgD alone induces cell proliferation. Analysis of IgD-deficient mice shows that the absence of IgD reduces the efficacy of B cell participation in immune responses. Further in vitro differences in antibody responses, immunological memory, and tolerance have also been described (Carsetti, R. et al., 1993. A role for immunoglobulin D: interference with tolerance induction. Eur. J. Immunol. 23: 168-178; Roes, J. et al., 1993. Immunoglobulin D (IgD)-deficient mice reveal an auxiliary receptor function for IgD in antigen-mediated recruitment of B cells. J Exp Med 177:45-55; and Kim, K. M. et al., 1992. Anti-IgM but not anti-IgD antibodies inhibit cell division of normal human mature B cells. J. Immunol. 148: 29-34).

### B Cell Receptor Activation

Signaling through the BCR plays an important role in both the generation of antibody and in the establishment of immunological tolerance. Furthermore, the outcome of B-cell receptor ligation on B-cell development and survival is influenced by multiple parameters. Immature B cells that bind self-antigen in the bone marrow are eliminated by apoptosis, and thereby antibodies to self are eliminated. In contrast, antigen binding on mature B cells results in activation, proliferation, anergy, or apoptosis, depending on the physical properties of the antigen itself, and costimuli provided by different components of the innate and acquired immunity. Therefore, the nature of the interaction between antigen and the B cell receptor (and the co-receptors) is an important consideration in the design of a vaccine immunogen. Specific antigen binding that leads to B cell activation is thought to induce a conformational change in the B cell receptor (BCR), and various models are proposed how early processes on the surface of B cells lead to antigen-induced B cell activation (Batista & Harwood, 2009. The who, how and where of antigen presentation to B cells. Nat Rev Immunol 9(1):15-27; Harwood & Batista, 2008. New Insights into the Early Molecular Events Underlying B Cell Activation. Immunity 28: 609-619; Gupta & DeFranco, 2007. Lipid rafts and B cell signaling. Semin. Cell Dev. Biol. 18: 616-626; Pierce, 2002. Lipid rafts and B-cell activation. Nat. Rev. Immunol. 2: 96-105; Reth, 2001. Oligomeric antigen receptors: A new view on signaling for the selection of lymphocytes. Trends Immunol. 22, 356-360; Reth, 1989. Antigen receptor tail clue. Nature 338, 383-384; Schamel & Reth, 2000. Monomeric and oligomeric complexes of the B cell antigen receptor. Immunity 13, 5-14; Sohn et al., 2006. Fluorescence resonance energy transfer in living cells reveals dynamic membrane changes in the initiation of B cell signaling. Proc Natl Acad Sci USA 103, 8143-8148; Engels et al., 2008. Conformational plasticity and navigation of signaling proteins in antigen-activated B lymphocytes. Adv Immunol 97:251-81). Surface immunoglobulin mediates signaling, and the strength of the signals transmitted depends on various factors, including affinity and avidity of antigen-immunoglobulin interaction. Different signal strengths induce qualitatively different signaling consequences; relatively weak signals may induce DNA synthesis; moderate signals may induce DNA synthesis with cell cycle arrest at the G2/M interphase; and intense signals may induce apoptotic cell death, and thus the concept of activation-induced cell death also applies to mature B lymphocytes (Mayumi et al. 1996. Negative signaling in B cells by surface immunoglobulins. J Allergy Clin Immunol. 98(6 Pt 2):S238-47; Berard et al., 1999. Activation sensitizes human memory B cells to B-cell receptor-induced apoptosis. Immunology 98(1):47-54.). Antigen-specific B cells are programmed shortly after antigen encounter to differentiate to long-lived plasma cells (“PC”), short-lived PCs, or B memory cells based on their intrinsic BCR affinity for antigen (Benson et al., 2007. Affinity of antigen encounter and other early B-cell signals determine B-cell fate. Current Opinion in Immunology 19:275-280).

B Cell Receptor Signaling is Required for B Cell Development and Maturation

A requirement for Igμ expression and B cell receptor expression in B cell survival has been demonstrated by conditional, CRE-inducible gene-targeting experiments. Loss of Igμ expression in peripheral B cells induces programmed cell death (Lam, Kuhn, & Rajewsky, 1997. In vivo ablation of surface immunoglobulin on mature B cells by inducible gene targeting results in rapid cell death. Cell 90: 1073-1083; Meffre & Nussenzweig, 2002. Deletion of immunoglobulin beta in developing B cells leads to cell death. Proc Natl Acad Sci USA 99(17):11334-11339). Similarly, loss of expression of Igβ causes developmental arrest and apotosis in immature B cells. Igβ knock-out mice were produced by the methods described in Gong & Nussenzweig, 1996 (Gong & Nussenzweig, 1996. Regulation of an early developmental checkpoint in the B cell pathway by Ig beta. Science 272(5260):411-4). To generate Igβ transgenic animals a bacterial artificial chromosome (BAC) containing the RAG1 and RAG 2 genes and all of the regulatory elements required to direct RAG expression in vivo was introduced, and Igβ cDNA was inserted at the RAG2 start codon in N-BAC by homologous recombination by the methods described in Meffre & Nussenzweig, 2002, Yu et al., 1999, and Misulovin et al., 2001 (Meffre & Nussenzweig, 2002. Deletion of immunoglobulin beta in developing B cells leads to cell death. Proc Natl Acad Sci USA 99(17):11334-11339; Yu et al., 1999. Continued RAG expression in late stages of B cell development and no apparent re-induction after immunization. Nature 400: 682-687; and Misulovin et al., 2001. A rapid method for targeted modification and screening of recombinant bacterial artificial chromosome. J. Immunol. Methods 257: 99-105).

### Immunogen Designs & Strategies for Vaccines Against Viruses

Identifying immunogens that interact with, and activate BCRs in such a way that B cells produce broadly neutralizing Ab's is critical in humoral vaccine design.

### Conformational Changes and Humoral Immune Responses to Viral Infection

Assembly of infectious virus particles involves many structural intermediates of proteins, and after virus binding to the host cell receptor, many structural and conformational changes are required for, and occur during, the processes of infection. The immune system responds to infection by a virus that displays multiple conformations with an array of neutralizing antibodies (NAbs). There are multiple examples of NAb-inhibited or NAb-induced conformational changes for many viruses. Antibody-mediated neutralization of virus through the induction of conformational changes has been demonstrated for non-enveloped and enveloped viruses. NAbs that bind influenza virus, poliovirus, rabies virus, rotavirus, and adenoviruses block conformational changes that are required for virus entry into the target cell (Emini et al., 1983. Bivalent attachment of antibody onto poliovirus leads to conformational alteration and neutralization. J. Virol. 48:547-550; Imai et al., 1998. Fusion of influenza virus with the endosomal membrane is inhibited by monoclonal antibodies to defined epitopes on the hemagglutinin. Virus Res. 53:129-139; Kida et al., 1985. Interference with a conformational change in the haemagglutinin molecule of influenza virus by antibodies as a possible neutralization mechanism. Vaccine 3:219-222; Raux et al., 1995. Monoclonal antibodies which recognize the acidic configuration of the rabies glycoprotein at the surface of the virion can be neutralizing. Virology 210:400-408; Wohlfart. 1988. Neutralization of adenoviruses: kinetics, stoichiometry, and mechanisms. J. Virol. 62: 2321-2328; Paredes et al., 2004. Conformational changes in Sindbis virions resulting from exposure to low pH and interactions with cells suggest that cell penetration may occur at the cell surface in the absence of membrane fusion. Virology 324:373-86; Schmaljohn et al., 1983. Protective monoclonal antibodies define maturational and pH-dependent antigenic changes in Sindbis virus E1 glycoprotein. Virology 130:144-54; Wetz et al., 1986. Neutralization of poliovirus by polyclonal antibodies requires binding of a single IgG molecule per virion. Arch Virol 91:207-20).

Ab-mediated binding occlusion or steric interference is the best understood mechanism of virus neutralization, but new evidence suggests that neutralization mechanisms can disrupt infection by interfering with the development of conformations of the virus particle required for any phase of the infection pathway (Crowe et al., 2001. Genetic and structural determinants of virus neutralizing antibodies. Immunol Res 23:135-45; Phinney & Brown. 2000. Sindbis virus glycoprotein E1 is divided into two discrete domains at amino acid 129 by disulfide bridge connections. J Virol 74:9313-6; Schmaljohn, A. L., K. M. Kokubun, and G. A. Cole. 1983. Protective monoclonal antibodies define maturational and pH-dependent antigenic changes in Sindbis virus E1 glycoprotein. Virology 130:144-54).

The structural biology of the influenza virus illustrates the importance of transient protein states for host cell infection; virus particle proteins undergo complex conformational changes induced by low pH to deliver the viral genome to the host cell (Edwards & Dimmock, 2001. A haemagglutinin (HA1)-specific FAb neutralizes influenza A virus by inhibiting fusion activity. Journal of General Virology 82:1387-1395; Symington et al., 1977. Infectious virus antibody complexes of sindbis virus. Infect Immun 15:720-5). Antibodies (Abs) that neutralize fusion activity of the influenza virus have been identified, whereby conformational changes to intermediate structures required for membrane fusion and infection are blocked (Edwards & Dimmock, 2001. A haemagglutinin (HA1)-specific FAb neutralizes influenza A virus by inhibiting fusion activity. Journal of General Virology 82:1387-1395; Imai et al., 1998. Fusion of influenza virus with the endosomal membrane is inhibited by monoclonal antibodies to defined epitopes on the hemagglutinin. Virus Research 53:129-139).

Abs against respiratory syncytial virus and rabies virus have been identified that neutralize virus infection by inducing conformational changes in virus particle structures (Crowe et al., 2001. Genetic and structural determinants of virus neutralizing antibodies. Immunol Res 23:135-45; Imai et al., 1998. Fusion of influenza virus with the endosomal membrane is inhibited by monoclonal antibodies to defined epitopes on the hemagglutinin. Virus Research 53:129-139; Irie & Kawai, 2005. Further studies on the mechanism of rabies virus neutralization by a viral glycoprotein-specific monoclonal antibody, #1-46-12. Microbiol Immunol 49:721-31).

Studies using poliovirus demonstrated that virus neutralization could occur with one or two Ab molecules per virion, and virus neutralization of rabies virus by one specific MAb was proposed to be mediated through the binding of a few Ab molecules per virion, producing conformational changes which were propagated throughout the particle in a neutralization cascade termed the “domino effect” (Wang et al., 2007. Infection of cells by Sindbis virus at low temperature. Virology 5; 362(2):461-7; Irie & Kawai, 2005. Further studies on the mechanism of rabies virus neutralization by a viral glycoprotein-specific monoclonal antibody, #1-46-12. Microbiol. Immunol. 49:721-731; Reading & Dimmock, 2007. Neutralization of animal virus infectivity by antibody. Arch. Virol. 152:1047-1059). In that system, MAb-induced conformational changes induced by ≦20 molecules bound to G proteins (about 600 trimeric spikes per virion) were proposed to spread to neighboring G proteins, resulting in the loss of the receptor binding conformation of the remaining proteins and neutralization of the virion (Irie & Kawai, 2002. Studies on the different conditions for rabies virus neutralization by monoclonal antibodies #1-46-12 and #7-1-9. J. Gen. Virol. 83:3045-3053). Previous virus neutralization models proposed for Venezuelan equine encephalomyelitis virus suggested that virus glycoprotein conformations could be altered to stabilize virus-cell receptor interactions disrupting infection or by inducing the formation of noninfectious immune complexes which were still able to attach to cells (Roehrig, 1988. In vitro mechanisms of monoclonal antibody neutralization of Alphaviruses. Virology 165:66-73).

Neutralization of human immunodeficiency virus (HIV) by monoclonal antibodies (MAbs) involves a similar mechanism that disrupts infection by Ab-mediated interference with one or more structural rearrangements required for fusion (Cardoso et al., 2005. Broadly neutralizing anti-HIV antibody 4E 10 recognizes a helical conformation of a highly conserved fusion associated motif in gp41. Immunity 22:163-73; Eckert & Kim, 2001. Mechanisms of viral membrane fusion and its inhibition. Annu Rev Biochem 70:777-810). Additionally, as described in greater detail below, there is evidence that HIV resists Ab-mediated neutralization through conformational masking of the conserved receptor-binding sites on the virus that require induced conformations in order to bind the receptors (Kwong et al., 2002. HIV-1 evades antibody-mediated neutralization through conformational masking of receptor binding sites. Nature 420:678-82).

Human Immunodeficiency Virus (HIV) Vaccines: Conformational Masking

The HIV envelope glycoproteins, gp120 and gp41, are the proteolytic products of the precursor protein, gp160. gp120 and gp41 form a non-covalent dimer, which trimerizes to form the viral spike (Env). gp41 is a transmembrane protein that mediates trimerization of the gp41-gp120 complex, and comprises the membrane fusion domain that affects fusion of the cellular and viral membranes, and entry of the HIV genetic material into host cells (Wu et al., 1996. CD4-induced interaction of primary HIV-1 gp120 glycoproteins with the chemokine receptor CCR-5. Nature 384: 179-83; Dalgleish et al., 1984. The CD4 (T4) antigen is an essential component of the receptor for the AIDS retrovirus. Nature 312: 763-7; Deng et al., 1996. Identification of a major co-receptor for primary isolates of HIV-1. Nature 381: 661-6; and Choe et al., 1996. The beta-chemokine receptors CCR3 and CCR5 facilitate infection by primary HIV-1 isolates. Cell 85: 1135-48). The exterior gp120 mediates trimerization at the apex of the spike via the V1/V2 loop, and receptor binding; the protein undergoes several entry-related conformational changes, first upon binding to the receptor, CD4, and subsequently upon interaction with a co-receptor, CCR5 or CXCR4 (Wyatt & Sodroski, 1998. The HIV-1 envelope glycoproteins: fusogens, antigens, and immunogens. Science 280: 1884-8; and Liu et al., 2008. Molecular architecture of native HIV-1 gp120 trimers. Nature 455: 109). Due to its position on the surface of viral particles, and to its exposure to cells of the immune system, the trimeric Env protein complex binds most of the NAbs identified to date (Wyatt et al., 1998. The antigenic structure of the HP/gp120 envelope glycoprotein. Nature 393: 705-11; the foregoing reference is incorporated in its entirety herein); therefore, the spike is the focus of much of HIV-1 vaccine immunogen design to elicit immune responses that produce NAbs.

In designing HIV immunogens, targeting the conserved regions of the HIV-1 spike has proven extremely difficult. The “conformational mask” model of immune evasion is among the most important mechanisms by which the “entry-functional” spike resists the binding of neutralizing antibodies (Kwong et al., 2002. HIV-1 evades antibody-mediated neutralization through conformational masking of receptor-binding sites. Nature; 420: 678-82; Phogat & Wyatt, 2007. Rational Modifications of HIV-1 Envelope Glycoproteins for Immunogen Design. Current Pharmaceutical Design 13, 213-227; both of the foregoing references are incorporated in their entirety herein). It is thought that an antibody that efficiently binds the “entry-functional” spike can neutralize the virus; Yang et al further demonstrate that binding of a single Ab per functional spike is sufficient for neutralization (Yang et al., 2005. Stoichiometry of antibody neutralization of human immunodeficiency virus type 1. J Virol 79: 3500-8; the foregoing reference is incorporated in its entirety herein). According to the conformational masking model, each trimeric, functional spike is a tightly packed sphere of variable protein elements covered by a contiguous glycan shield, a structure that has evolved by to prevent binding of most antibodies (Wei et al., 2003. Antibody neutralization and escape by HIV-1. Nature 422: 307-12; Myers, Maclnnes, & Korber. 1992. The emergence of simian/human immunodeficiency viruses. AIDS Res Hum Retroviruses 8: 373-86; Kuiken C L, et al., Eds. 2002, HIV Sequence Compendium. Theoretical Biology and Biophysics Group: Los Alamos National Laboratory, Los Alamos; Phogat & Wyatt, 2007. Rational Modifications of HIV-1 Envelope Glycoproteins for Immunogen Design. Current Pharmaceutical Design 13, 213-227; each of the foregoing references is incorporated in its entirety herein).

### Knock Out, and Transexpression of Immunoglobulin Molecules in B Cells

Several studies have been published that address the role of the molecules of the B cell receptor, including membrane-bound IgM and IgD, and immunoglobulin alpha and beta, also by knock-out and/or trans expression in transgenic mice (see, for example, Pelanda et al., 2002. B cell progenitors are arrested in maturation but have intact VDJ recombination in the absence of Ig-alpha and Ig-beta. J Immunol 169(2):865-72; Meffre et al., 2000. Antibody regulation of B cell development. Nat Immunol 1(5):379-85; Buhl et al., 2000. B-cell antigen receptor competence regulates B-lymphocyte selection and survival. Immunol Rev 176:141-53; Nussenzweig et al., 1987. Allelic exclusion in transgenic mice that express the membrane form of immunoglobulin mu. Science 236: 816-819. Reth, 1989. Antigen receptor tail clue. Nature 338: 383-384; Nalcamura et al., 1992. Heterogeneity of immunoglobulin-associated molecules on human B cells identified by monoclonal antibodies. Proc. Natl. Acad. Sci. USA 89: 8522-8526; and the remaining references of this and the following paragraph). A requirement for Igμ expression and B cell receptor expression in B cell survival has been demonstrated by conditional, CRE-inducible gene-targeting experiments. Loss of Igμ expression in peripheral B cells induces programmed cell death (Lam, Kuhn, & Rajewsky, 1997. In vivo ablation of surface immunoglobulin on mature B cells by inducible gene targeting results in rapid cell death. Cell 90: 1073-1083; Meffre & Nussenzweig, 2002. Deletion of immunoglobulin beta in developing B cells leads to cell death. Proc Natl Acad Sci USA 99(17):11334-11339). Similarly, loss of expression of Igβ causes developmental arrest and apotosis in immature B cells. Igb knock-out mice were produced by the methods described in Gong & Nussenzweig, 1996 (Gong & Nussenzweig, 1996. Regulation of an early developmental checkpoint in the B cell pathway by Ig beta. Science 272(5260):411-4). To generate Igβ transgenic animals a bacterial artificial chromosome (BAC) containing the RAG1 and RAG 2 genes and all of the regulatory elements required to direct RAG expression in vivo was introduced, and Igβ cDNA was inserted at the RAG2 start codon in N-BAC by homologous recombination by the methods described in Meffre & Nussenzweig, 2002, Yu et al., 1999, and Misulovin et al., 2001 (Meffre & Nussenzweig, 2002. Deletion of immunoglobulin beta in developing B cells leads to cell death. Proc Natl Acad Sci USA 99(17):11334-11339; Yu et al., 1999. Continued RAG expression in late stages of B cell development and no apparent re-induction after immunization. Nature 400: 682-687; and Misulovin et al., 2001. A rapid method for targeted modification and screening of recombinant bacterial artificial chromosome. J. Immunol. Methods 257: 99-105).

### Existence of Neutralizing and Broadly Neutralizing Antibodies

As described above, many antibodies have been identified that bind and neutralize, as non-limiting examples, influenza virus, poliovirus, rabies virus, rotavirus, and adenoviruses (Emini et al., 1983. Bivalent attachment of antibody onto poliovirus leads to conformational alteration and neutralization. J. Virol. 48:547-550; Imai et al., 1998. Fusion of influenza virus with the endosomal membrane is inhibited by monoclonal antibodies to defined epitopes on the hemagglutinin. Virus Res. 53:129-139; Kida et al., 1985. Interference with a conformational change in the haemagglutinin molecule of influenza virus by antibodies as a possible neutralization mechanism. Vaccine 3:219-222; Raux et al., 1995. Monoclonal antibodies which recognize the acidic configuration of the rabies glycoprotein at the surface of the virion can be neutralizing. Virology 210:400-408; Wohlfart. 1988. Neutralization of adenoviruses: kinetics, stoichiometry, and mechanisms. J. Virol. 62: 2321-2328; Paredes et al., 2004. Conformational changes in Sindbis virions resulting from exposure to low pH and interactions with cells suggest that cell penetration may occur at the cell surface in the absence of membrane fusion. Virology 324:373-86; Schmaljohn et al., 1983. Protective monoclonal antibodies define maturational and pH-dependent antigenic changes in Sindbis virus E1 glycoprotein. Virology 130:144-54; Wetz et al., 1986. Neutralization of poliovirus by polyclonal antibodies requires binding of a single IgG molecule per virion. Arch Virol 91:207-20). As described further above, some antibodies that induce conformational changes are highly potent due to the “domino effect” of conformational changes of proteins on the surface of the viron (Wang et al., 2007. Infection of cells by Sindbis virus at low temperature. Virology 362 (2): 461-467; Irie & Kawai, 2005. Further studies on the mechanism of rabies virus neutralization by a viral glycoprotein-specific monoclonal antibody, #1-46-12. Microbiol. Immunol. 49:721-731; Reading & Dimmock, 2007. Neutralization of animal virus infectivity by antibody. Arch. Virol. 152:1047-1059). In that system, MAb-induced conformational changes induced by 520 molecules bound to G proteins (about 600 trimeric spikes per virion) were proposed to spread to neighboring G proteins, resulting in the loss of the receptor binding conformation of the remaining proteins and neutralization of the virion (Irie & Kawai, 2002. Studies on the different conditions for rabies virus neutralization by monoclonal antibodies #1-46-12 and #7-1-9. J. Gen. Virol. 83:3045-3053).

The existence of antibodies that neutralize by introducing conformational changes indicates that in some instances an antigen stabilized in a particular conformation, such as, for example, but not limited to, the conformation of the antigen to which binding of a particular NAb induces change, is more likely to elicit a humoral immune response that leads to the production of such antibodies. Identification of such antigens would therefore be of significant value.

### Neutralizing and Broadly Neutralizing Antibodies to the HIV Spike Proteins

Significant progress has been made to date relating to the understanding of HIV proteins and vaccine development (see, for example, Wyatt & Sodroski, 1998. The HIV-1 envelope glycoproteins: fusogens, antigens, and immunogens. Science 280: 1884-88; Wu et al., 1996. CD4-induced interaction of primary HIV-1 gp120 glycoproteins with the chemokine receptor CCR-5. Nature 384: 179-83; Dalgleish et al., 1984. The CD4 (T4) antigen is an essential component of the receptor for the AIDS retrovirus. Nature 312: 763-67; Deng et al., 1996. Identification of a major co-receptor for primary isolates of HIV-1. Nature 381: 661-66; Choe et al., 1996. The beta-chemokine receptors CCR3 and CCR5 facilitate infection by primary HIV-1 isolates. Cell 85: 1135-48; Wyatt et al., 1998. The antigenic structure of the HIV gp120 envelope glycoprotein. Nature 393: 705-11; Burton et al., 2004. HIV vaccine design and the neutralizing antibody problem. Nat Immunol 5: 233-36; Zwick et al., 2001. Broadly neutralizing antibodies targeted to the membrane-proximal external region of human immunodeficiency virus type 1 glycoprotein gp41. J Virol 75: 10892-905; Hartley et al., 2005. V3: HIV's switchhitter. AIDS Res Hum Retroviruses 21: 171-89; Gorny et al. 2002. Human monoclonal antibodies specific for conformationsensitive epitopes of V3 neutralize human immunodeficiency virus type 1 primary isolates from various clades. J Virol 76: 9035-45; Huang et al., 2005. Structure of a V3-containing HIV-1 gp120 core. Science 310: 1025-28; Rizzuto & Sodroski, 2000. Fine definition of a conserved CCR5-binding region on the human immunodeficiency virus type 1 glycoprotein 120. AIDS Res Hum Retroviruses 16: 741-49; Rizzuto et al., 1998. A conserved HIV gp120 glycoprotein structure involved in chemokine receptor binding. Science 280: 1949-53; Labrijn et al., 2003. Access of antibody molecules to the conserved coreceptor binding site on glycoprotein gp120 is sterically restricted on primary human immunodeficiency virus type 1. J Virol 77: 10557-65; Moulard et al., 2002. Broadly cross-reactive HIV-1-neutralizing human monoclonal Fab selected for binding to gp120-CD4-CCR5 complexes. Proc Natl Acad Sci USA 99: 6913-18; Decker et al., 2005. Antigenic conservation and immunogenicity of the HIV coreceptor binding site. J Exp Med 201: 1407-19; Kwong et al. 2000. Structures of HIV-1 gp120 envelope glycoproteins from laboratory-adapted and primary isolates. Structure Fold Des 8: 1329-39; Kwong et al., 1998. Structure of an HIV gp120 envelope glycoprotein in complex with the CD4 receptor and a neutralizing human antibody. Nature 393: 648-59; Kwong et al., 2002. HIV-1 evades antibody-mediated neutralization through conformational masking of receptor-binding sites. Nature 420: 678-82; Wei et al., 2003. Antibody neutralization and escape by HIV-1. Nature 422: 307-12; Myers et al., 1992. The emergence of simian/human immunodeficiency viruses. AIDS Res Hum Retroviruses; 8: 373-86; Kuiken et al., 2002. Eds. HIV Sequence Compendium. Theoretical Biology and Biophysics Group: Los Alamos National Laboratory, Los Alamos; Yang et al., 2005. Stoichiometry of antibody neutralization of human immunodeficiency virus type 1. J Virol 79: 3500-08; Par en et al., 1997. HIV-1 antibody—debris or virion? Nat Med 3: 366-67; Chertova et al. 2002. Envelope glycoprotein incorporation, not shedding of surface envelope glycoprotein (gp120/SU), Is the primary determinant of SU content of purified human immunodeficiency virus type 1 and simian immunodeficiency virus. J Virol 76: 5315-25; Myszka et al., 2000. Energetics of the HIV gp120-CD4 binding reaction. Proc Natl Acad Sci USA 97: 9026-31; Chen et al., 2005. Structure of an unliganded simian immunodeficiency virus gp120 core. Nature 433: 834-41; Barnett et al., 1997. Vaccination with HIV-1 gp120 DNA induces immune responses that are boosted by a recombinant gp120 protein subunit. Vaccine 15: 869-73; Belshe et al. 1998. Induction of immune responses to HIV-1 by canarypox virus (ALVAC) HIV-1 and gp120 SF-2 recombinant vaccines in uninfected volunteers. NIAID AIDS Vaccine Evaluation Group. Aids 12: 2407-15; Berman et al. 1990. Protection of chimpanzees from infection by HIV-1 after vaccination with recombinant glycoprotein gp120 but not gp160. Nature 345: 622-25; Connor et al., 1998. Immunological and virological analyses of persons infected by human immunodeficiency virus type 1 while participating in trials of recombinant gp120 subunit vaccines. J Virol 72: 1552-76; Mascola et al. 1996. Immunization with envelope subunit vaccineproducts elicits neutralizing antibodies against laboratory-adapted but not primary isolates of human immunodeficiency virus type 1. The National Institute of Allergy and Infectious Diseases AIDS Vaccine Evaluation Group. J Infect Dis 173: 340-48; Wrin et al., 1995. Adaptation to persistent growth in the H9 cell line renders a primary isolate of human immunodeficiency virus type 1 sensitive to neutralization by vaccine sera. J Virol 69: 39-48; Gilbert et al., 2005. Correlation between immunologic responses to a recombinant glycoprotein 120 vaccine and incidence of HIV-1 infection in a phase 3 HIV-1 preventive vaccine trial. J Infect D is 191: 666-77; Graham & Mascola, 2005. Lessons from failure—preparing for future HIV-1 vaccine efficacy trials. J Infect Dis 191: 647-49; Burton et al., 2005. Antibody vs. HIV in a clash of evolutionary titans. Proc Natl Acad Sci USA 102: 14943-48; Koff et al., 2006. HIV vaccine design: insights from live attenuated SIV vaccines. Nat Immunol 7: 19-23; Arthur et al., 1995. Macaques immunized with HLA-DR are protected from challenge with simian immunodeficiency virus. J Virol 69: 3117-24; Lifson et al., 2004. Evaluation of the safety, immunogenicity, and protective efficacy of whole inactivated simian immunodeficiency virus (SIV) vaccines with conformationally and functionally intact envelope glycoproteins. AIDS Res Hum Retroviruses 20: 772-87; Rossio et al., 1998. Inactivation of human immunodeficiency virus type 1 infectivity with preservation of conformational and functional integrity of virion surface proteins. J Virol 72: 7992-8001; Deml et al., 2005. Recombinant HIV-1 Pr55gag virus-like particles: potent stimulators of innate and acquired immune responses. Mol Immunol 42: 259-77; Poon et al., 2005. Formaldehyde-treated, heat-inactivated virions with increased human immunodeficiency virus type 1 env can be used to induce high-titer neutralizing antibody responses. J Virol 79: 10210-17; Poon et al., 2005. Induction of humoral immune responses following vaccination with envelope-containing, formaldehyde-treated, thermally inactivated human immunodeficiency virus type 1. J Virol 79: 4927-35; Doan et al., 2005. Virus-like particles as HIV-1 vaccines. Rev Med Virol 15: 75-88; Fouts et al., 2002. Crosslinked HIV-1 envelope-CD4 receptor complexes elicit broadly cross-reactive neutralizing antibodies in rhesus macaques. Proc Natl Acad Sci USA 99: 11842-47; Varadarajan et al., 2005. Characterization of gp120 and its single-chain derivatives, gp120-CD4D12 and gp120-M9: implications for targeting the CD4i epitope in human immunodeficiency virus vaccine design. J Virol 79: 1713-23; Liao et al., 2004. Immunogenicity of constrained monoclonal antibody A32-human immunodeficiency virus (HIV) Env gp120 complexes compared to that of recombinant HIV type 1 gp120 envelope glycoproteins. J Virol 78: 5270-78; Hoffman et al., 1999. Stable exposure of the coreceptor-binding site in a CD4-independent HIV-1 envelope protein. Proc Natl Acad Sci USA 96: 6359-64).

To date, only four rare NAbs have been identified that neutralize HIV broadly across strains and clades, hereinafter referred to as broadly neutralizing antibodies (“BNAbs”; Burton et al., 2004. HIV vaccine design and the neutralizing antibody problem. Nat Immunol; 5: 233-6; the foregoing reference is incorporated in its entirety herein); efforts to identify additional BNAbs are ongoing. On gp120, the antibody IgG1 b12 binds the CD4 binding site (CD4BS) of the spike and competes with CD4 binding. The other gp120 BNAb, 2G12, binds to a cluster of glycans on the surface of the gp120 protein. The gp41-directed antibodies, 2F5 and 4E10, bind hydrophobic epitopes within a region close to the viral membrane that is highly conserved across clades (Zwick et al., 2001. Broadly neutralizing antibodies targeted to the membrane-proximal external region of human immunodeficiency virus type 1 glycoprotein gp41. J Virol; 75: 10892-905; the foregoing reference is incorporated in its entirety herein). Most antibodies to the immunodominant variable loop 3 (V3) of gp120 are strain-restricted, with the exception of the 447-52D antibody that displays a certain degree breadth of neutralization (Hartley et al., 2005. V3: HIV's switchhitter. AIDS Res Hum Retroviruses; 21: 171-89; Gomy et al., 2002. Human monoclonal antibodies specific for conformationsensitive epitopes of V3 neutralize human immunodeficiency virus type 1 primary isolates from various clades. J Virol; 76: 9035-45; both of the foregoing references are incorporated in its entirety herein).

Another group of characterized antibodies detected at relatively high levels in patient sera recognizes the highly conserved co-receptor binding site on the gp120 core that becomes exposed upon binding of the spike to the CD4 receptor; these Abs are hence termed CD4-induced (“CD4i”). In primary isolates, however, these epitopes are not accessible prior to binding of the spike to the CD4 receptor, and only FAb fragments or single chain constructs of these antibodies effectively neutralize primary isolates, presumably because the epitopes are occluded due to steric hindrance (Labrijn et al., 2003. Access of antibody molecules to the conserved coreceptor binding site on glycoprotein gp120 is sterically restricted on primary human immunodeficiency virus type 1. J Virol 77: 10557-65; Moulard et al., 2002. Broadly cross-reactive HIV-1-neutralizing human monoclonal Fab selected for binding to gp120-CD4-CCR5 complexes. Proc Natl Acad Sci USA 99: 6913-8; Decker et al., 2005. Antigenic conservation and immunogenicity of the HIV coreceptor binding site. J Exp Med 201: 1407-19; each of the foregoing references is incorporated in its entirety herein). It is likely that occlusion of this conserved site on primary isolates is the result of strong in vivo selection pressure, and/or that virus particles with spike structures that more readily display these epitopes are not capable of infecting host cells productively.

Antibodies against variable elements are capable of neutralizing HIV virus, but not with a significant degree of breadth across clades, and hence such antibodies represent inadequate responses for a vaccine. To date, gp120 immunogens characterized in immunogenicity tests have been ineffectual in eliciting BNAbs; it is thought this may be due to the monomeric proteins lack of shielding or masking properties (Phogat & Wyatt, 2007. Rational Modifications of HIV-1 Envelope Glycoproteins for Immunogen Design. Current Pharmaceutical Design 13, 213-227; Barnett et al., 1997. Vaccination with HIV-1 gp120 DNA induces immune responses that are boosted by a recombinant gp120 protein subunit. Vaccine 15: 869-73; Belshe et al., 1998. Induction of immune responses to HIV-1 by canarypox virus (ALVAC) HIV-1 and gp120 SF-2 recombinant vaccines in uninfected volunteers. NIAID AIDS Vaccine Evaluation Group. Aids 12: 2407-15; Berman et al., 1990. Protection of chimpanzees from infection by HIV-1 after vaccination with recombinant glycoprotein gp120 but not gp160. Nature 345: 622-5; Connor et al., 1998. Immunological and virological analyses of persons infected by human immunodeficiency virus type 1 while participating in trials of recombinant gp120 subunit vaccines. J Virol 72: 1552-76; Mascola et al., 1996. Immunization with envelope subunit vaccineproducts elicits neutralizing antibodies against laboratory-adapted but not primary isolates of human immunodeficiency virus type 1. The National Institute of Allergy and Infectious Diseases AIDS Vaccine Evaluation Group. J Infect Dis 173: 340-8; Wrin et al., 1995. Adaptation to persistent growth in the H9 cell line renders a primary isolate of human immunodeficiency virus type 1 sensitive to neutralization by vaccine sera. J Virol 69: 39-48; each of the foregoing references is incorporated in its entirety herein).

Patient sera with broad neutralizing activities in response to natural infection argue that it is possible to elicit antibody responses to HIV that are broadly neutralizing, and the four characterized BNAs and their epitopes provide the most important information available in humoral immunogen design. Ongoing efforts to identify additional epitopes targeted by BNAs present in broadly neutralizing patient sera may yield addition information that can be used to hone future immunogen design efforts. Identification of antigens that induce humor immune responses to these epitopes in such a way that the resultant antibodies are broadly neutralizing across strains and Glades would be of significant value.

Furthermore, in HIV-infected patients with broadly neutralizing antibodies and low to intermediate viral loads, B-cell memory response to gp140 is composed of up to 50 independent clones expressing high affinity neutralizing antibodies to the gp120 variable loops, the CD4-binding site, the co-receptor-binding site, and to another neutralizing epitope in the same region of gp120 as the CD4-binding site. Thus, the IgG memory B-cell compartment is comprised of multiple clonal responses with neutralizing activity directed against several epitopes on gp120 (Scheid et al., 2009. Broad diversity of neutralizing antibodies isolated from memory B cells in HIV-infected individuals. Nature Nature 458(7238):584-5).

Immunogen Design in HIV Vaccine Research and Development Presentation of the native envelope trimer to the immune system in the context of live attenuated virus, inactivated virus or of virus like particles (VLPs) has long been considered a promising approach, but has suffered significant set-backs; subunit immunogenicity studies largely focused on gp120 and peptides derived from the immunodominant V3 loop have also not met with success (Phogat & Wyatt, 2007. Rational Modifications of HIV-1 Envelope Glycoproteins for Immunogen Design. Current Pharmaceutical Design 13, 213-227; Gilbert et al., 2005. Correlation between immunologic responses to a recombinant glycoprotein 120 vaccine and incidence of HIV-1 infection in a phase 3 HIV-1 preventive vaccine trial. J Infect Dis 191: 666-77; Koff et al., 2006. HIV vaccine design: insights from live attenuated SIV vaccines. Nat Immunol 7: 19-23; Arthur et al., 1995. Macaques immunized with HLA-DR are protected from challenge with simian immunodeficiency virus. J Virol 69: 3117-24; Deml et al., 2005. Recombinant HIV-1 Pr55gag virus-like particles: potent stimulators of innate and acquired immune responses. Mol Immunol 42: 259-77; Poon et al., 2005. Formaldehyde-treated, heat-inactivated virions with increased human immunodeficiency virus type 1 env can be used to induce high-titer neutralizing antibody responses. J Virol 79: 10210-7; Poon et al., 2005. Induction of humoral immune responses following vaccination with envelope-containing, formaldehyde-treated, thermally inactivated human immunodeficiency virus type 1. J Virol 79: 4927-35; Doan et al., 2005. Virus-like particles as HIV-1 vaccines. Rev Med Virol 15: 75-88). Therefore, the field of humoral HIV immunogen design is focusing much of its efforts on trimeric Env constructs designed structurally more closely to mimic the functional spike, with the goal of eliciting immune responses that generate antibodies of the kind known to neutralize HIV within and across strains and clades.

Because there is very high degree of variability of Env residues within and across HIV clades, and it is thought that the best way to generate a broadly protective humoral immune response may be to target antibody responses to the conserved receptor binding sites on the Env protein complex, including the region of the gp120 core that interacts with the co-receptor and overlaps with the binding site of the well characterized 17b BNAb. Such studies have already shown varying levels of success, and efforts toward this goal are continuing (Phogat & Wyatt, 2007. Rational Modifications of HIV-1 Envelope Glycoproteins for Immunogen Design. Current Pharmaceutical Design 13, 213-227; Rizzuto & Sodroski, 2000. Fine definition of a conserved CCRS-binding region on the human immunodeficiency virus type 1 glycoprotein 120. AIDS Res Hum Retroviruses 16: 741-9; Rizzuto et al., 1998. A conserved HIV gp120 glycoprotein structure involved in chemokine receptor binding. Science 280: 1949-53; Fouts et al., 2002. Crosslinked HIV-1 envelope-CD4 receptor complexes elicit broadly cross-reactive neutralizing antibodies in rhesus macaques. Proc Natl Acad Sci USA 99: 11842-7; Varadarajan et al., 2005. Characterization of gp120 and its single-chain derivatives, gp120-CD4D12 and gp120-M9: implications for targeting the CD4i epitope in human immunodeficiency virus vaccine design. J Virol 79: 1713-23; Liao et al., 2004. Immunogenicity of constrained monoclonal antibody A32-human immunodeficiency virus (HIV) Env gp120 complexes compared to that of recombinant HIV type 1 gp120 envelope glycoproteins. J Virol 78: 5270-8; Hoffman et al., 1999. Stable exposure of the coreceptor-binding site in a CD4-independent HIV-1 envelope protein. Proc Natl Acad Sci USA 96: 6359-64). Several rational protein design strategies have been devised utilizing Env-based subunits to obtain immunogens that might better elicit broadly BNAbs:
(i) Design of Env derivatives that mimic desirable properties of the gp120:gp41 native trimeric spike on the virus;
(ii) Structure-based designs to stabilize gp120 in particular conformations; and
(iii) Structure-based designs of immunogens that expose otherwise cryptic epitopes

It is thought that for the development of HIV immunogens such new structures may represent promising opportunities to target immune responses to particular elements of Env that are demonstrated to be capable of generating BNAbs in vivo.

### Immunogen Evaluation

In the design of immunogens, it is necessary to assay for the success of a particular approach. Where possible, such assays are simple, not time consuming; in vitro assays are therefore preferable to in vivo assays. However, success can ultimately only be shown in vivo, where an immunogen generates an immune response to a pathogen or antigen that is therapeutic or protective. The closer the in vitro assay mimics the processes that are involved in vivo, however, the more accurate and reliable the in vitro assay is.

Given the existence of characterized BNAbs, and the resulting insight that antigens capable of eliciting immune responses that yield such antibodies must have existed in vivo, it is possible, and in the case of HIV vaccine research and development, significant efforts are being invested, to leverage the information gleaned from these molecules and their binding sites on the Env complex, to identify protein constructs capable of eliciting similar responses.

### Immunogenic Analysis

Immunogenic analyses, i.e. the determination of whether the engineered proteins or constructs used as immunogens generate antibodies that bind to specific antigenic structures, are performed by immunizing animals, such as for example, rabbits, rodents, or primates, and evaluating the resulting sera, for example, by ELISA and immunoprecipitation (Dey et al., 2007. Characterization of Human Immunodeficiency Virus Type 1 Monomeric and Trimeric gp120 Glycoproteins Stabilized in the CD4-Bound State: Antigenicity, Biophysics, and Immunogenicity. Virol 81(11): 5579-5593; Beddows et al., 2007. A comparative immunogenicity study in rabbits of disulfide-stabilized proteolytically cleaved, soluble trimeric human immunodeficiency virus type 1 gp140, trimeric cleavage-defective gp140 and momomeric gp120. Virol 360: 329-340).

### Neutralization Assays

Neutralization assays, i.e. the determination of whether antibodies or antisera generated by immunization of animals have viral neutralizing activity, are performed by incubating viral constructs with antisera and assaying for viral uptake by, and/or infection of, host cells, as described in detail by Dey et al. 2007 (Dey et al., 2007. Characterization of Human Immunodeficiency Virus Type 1 Monomeric and Trimeric gp120 Glycoproteins Stabilized in the CD4-Bound State: Antigenicity, Biophysics, and Immunogenicity. J Virol 81(11): 5579-5593) and Beddows et al., 2006 (Beddows et al., 2007. A comparative immunogenicity study in rabbits of disulfide-stabilized proteolytically cleaved, soluble trimeric human immunodeficiency virus type 1 gp140, trimeric cleavage-defective gp140 and momomeric gp120. Virol 360: 329-340).

### Antigenic Studies

Antigenic analyses, i.e. the determination of whether an engineered protein or construct binds specific antibodies, can be applied to determine whether one or more BNAb binds to a protein construct, and what certain binding characteristics of the antigen-receptor interactions are. This information is particularly relevant information that can be obtained in vitro, as antigen binding to the antigen binding site of the surface immunoglobulin of a B cell capable of producing and secreting antibodies is a requisite for B cell activation and maturation into plasma and memory cells. Methods by which antigenic analyses are performed are describe in detail, for example, in Dey et al. 2007 (Dey et al., 2007. Characterization of Human Immunodeficiency Virus Type 1 Monomeric and Trimeric gp120 Glycoproteins Stabilized in the CD4-Bound State: Antigenicity, Biophysics, and Immunogenicity. J Virol 81(11): 5579-5593), Binley et al., 2000 (Binley et al., 2000. A Recombinant Human Immunodeficiency Virus Type 1 Envelope Glycoprotein Complex Stabilized by an Intermolecular Disulfide Bond between the gp120 and gp41 Subunits Is an Antigenic Mimic of the Trimeric Virion-Associated Structure. J Virol 74(2): 627-643), Pancera et al., 2005 (Pancera et al., 2005. Soluble Mimetics of Human Immunodeficiency Virus Type 1 Viral Spikes Produced by Replacement of the Native Trimerization Domain with a Heterologous Trimerization Motif: Characterization and Ligand Binding Analysis. J Virol 79(15): 9954-9969), and Beddows et al., 2006 (Beddows et al., 2006. Construction and Characterization of Suluble, Cleaved, and Stabilized Trimeric Env proteins Based on HIV Type 1 env Subtype A. AIDS Res Hum Retroviruses 22(6): 569-579).

### Utility of an HTP-Compatible Assay that Reports BCR Activation Limitations of Protein Modeling in Vaccine Design

As the field of modeling and predicting protein structures and protein-protein interactions evolves, it may become possible to design protein-based immunogens that interact with particular BCRs in such a way that B cell activation is achieved in vivo for the purpose of vaccine immunogen design. However, to date, there are significant limitations in the art of molecular modeling, particularly with regard to protein-protein interactions, that render rational immunogen design ineffectual, and furthermore, many specific characteristics of antigen-receptor interactions required to induce B cell maturation to antibody secreting blast and memory cells remain unclear, and it is thus remains difficult to model antigens rationally for the purpose of designing vaccine immunogens (Risueño et al. 2008. Conformational model. Adv Exp Med Biol 640:103-12; Pierre Boudinot, 2008. New perspectives for large-scale repertoire analysis of immune receptors. Molecular Immunology 45: 2437-2445; Chen & Brooks, 2008. Implicit modeling of nonpolar solvation for simulating protein folding and conformational transitions. Phys Chem Chem Phys 10, 471-481; Kiel et al., 2008. Analyzing Protein Interaction Networks Using Structural Information. Annu Rev Biochem 77:415-441; Wang et al, 2001. Biomolecular Simulations: Recent Developments in Force Fields, Simulations of Enzyme Catalysis, Protein-Ligand, Protein-Protein, and Protein-Nucleic Acid Noncovalent Interactions. Annu Rev Biophys Biomol Struct 30:211-43; Van Regenmortel, 1989. Structural and functional approaches to the study of protein antigenicity. Immunol Today 10(8):266-72).

### An Assay that Reports Immogen-Induced Activation of Broadly Neutralizing B Cell Receptors

An assay that reports immogen-induced activation of B cell receptors comprising the antigen binding sites of antibodies known to possess broadly protective characteristics, stimulation of down-stream signaling, and/or B cell differentiation into broadly protective antibody secreting blast and memory cells would allow researchers to screen large numbers or libraries of peptides, mimetopes, proteins, mutations and/or constructs in vitro, accumulate useful, otherwise difficult to obtain information about the structure of the immunogen, and identify of one or more particular peptide, mimetope, protein, mutation, set of mutations, or construct(s) that triggers broadly protective humoral immune responses. Such high-through-put analysis of one or more libraries of mutations, sets of mutants, or constructs of a particular immunogen would help to circumvent the need to understand in detail the mechanism(s) by which a particular immunogen or a mutant, set of mutants, or construct of such an immunogen produces productive immune responses in patients, and/or the need to predict whether or how an engineered change to an immunogen would have an effect or impact on such a mechanism.

The initial signal strength resulting from B cell activation is the most important Ag-specific determinant of the nature of B cell responses in vivo. The total signal strength in a B cell following Ag binding, receptor internalization, antigen processing, and MHCII display, is made up of signals resulting from BCR activation and T cell help. The BCR signal is predominant, and drives naïve mature B cells to differentiate into short- and love-lived plasma cells and memory B cells (Benson et al., 2007. Curr Opin Immunol 19:275-280). Engineering T cell epitopes that support B cell differentiation following Ag-mediated activation can be accomplished by engineering effective T cell epitopes into the structures of immunogens that preserve the structural integrity of the relevant epitopes. Effective technologies to accomplish this are currently being commercialized (see, for example, EpiVax), and methods to enhance T cell help with proinflammatory cytokines and adjuvants are being further explored (Maue et al. 2009. J. Immunol. 2009 May 15; 182(10):6129-35). In vitro, the effect of T cell help can be mimicked by adding CD40 ligand and cytokines.

The characteristics of the interactions between antigen and BCR immunoglobulin that determine BCR-mediated signal strength are complex and currently not well understood, particularly in such cases like the characterized epitopes on HIV Env that can give rise to bnAbs and that are recessed and wedged between the various structural elements of the protein, its glycan shield, and the viral membrane. Crucially, an HTP assay that efficiently, reliably, and directly reports the signal strength resulting from antigen binding to the BCRs (comprising the heavy and light chains of characterized antibodies with broadly protective characteristics) would circumvent the need (i) to understand in detail the structural mechanism(s) by which immunogens induce BCR-mediated signals, and (ii) to predict whether or how an engineered change to an immunogen would affect such a mechanism. Such an HTP assay would allow researchers to:
Rapidly and inexpensively analyze libraries of peptides, mimetopes, proteins, mutations and/or constructs in vitro, and accumulate useful, otherwise difficult to obtain information;
Screen for molecules that trigger BCR signaling in B cells, and distinguish between signals that:
   Lead to apoptotic vs. proliferative responses;
   Lead to differentiation into short- and long-lived plasma cells and memory B cells.

In addition to speeding immunogen identification, iterative application of this methodology may enable significantly more effective approaches to immunogen design.

### 3. SUMMARY OF THE INVENTION

This invention provides a method for screening pathogenic viral envelope proteins and protein complexes to identify protein constructs with enhanced effectiveness as vaccine immunogens. The method is carried out by (i) expressing of a membrane-bound IgM and/or IgD isotype of an antibody that has the same binding activity and specificity of an antibody that is known, or identified, to bind and neutralize the targeted virus, and that has the capacity to activate signaling pathways down-stream of B cell receptor ligand binding and activation (“modified neutralizing antibody”), (ii) exposing the cell to antigen, and (iii) assay for signaling downstream of B cell receptor activation. In one particular embodiment, the present invention provides a method by which signaling down-stream of activation of a BCR comprising the modified neutralizing antibody is assayed in primary cells of a transgenic animal expressing the modified neutralizing antibody. In another embodiment, the present invention provides a method by which signaling down-stream of activation of a BCR comprising the modified neutralizing antibody is assayed in primary cells transiently transfected with an expression vector directing transcription and translation of a gene encoding the modified neutralizing antibody. In another embodiment, the present invention provides a method by which signaling down-stream of activation of a BCR comprising the modified neutralizing antibody is assayed in conditionally immortalized cells (described below), transiently of stably transfected with an expression vector directing transcription and translation of a gene encoding the modified neutralizing antibody. In another embodiment of the invention, expression of endogenous immunoglobulin heavy and light chains are knocked out or knocked down. In another embodiment, the present invention provides a method by which signaling down-stream of activation of a BCR comprising the modified neutralizing antibody is assayed by analyzing the properties of cytoplasmic signaling molecules and complexes. In another embodiment, the present invention provides a method by which signaling down-stream of activation of a BCR comprising the modified neutralizing antibody is assayed by measuring transcription rates of a reporter gene that is under transcriptional regulation of a promoter that is responsive to a transcription factor that is itself up- or down-regulated in response to BCR activation.

The present invention also provides the antigens identified using the assay described herein, and neutralizing antibodies derived by immunization with the antigens identified using the assay described herein.

### 4. BRIEF DESCRIPTION OF THE FIGURES

The present invention may be understood more fully by reference to the following detailed description, illustrative examples of specific embodiments and the appended figures.

FIG. 1. Live cells were gated based on forward and side scatter (A and C). Cells were stained with mouse anti-chicken IgM and IgM⁺ cells were detected with FITC conjugated anti-mouse secondary antibody (B and D).

FIG. 2. Ca⁺⁺ influx assayed in response to ionomycin treatment and BCR crosslinking in DT40 cells. (A) DT40 cells were loaded with Calcium 4 and stimulated with ionomycin (1.5 ng-20 ug). Ca⁺⁺ influx was monitored by fluorescence at 0.5-second intervals over a 6 minute time course. (B) DT40 cells expressing surface-bound IgM were loaded with Calcium 4 and stimulated with ionomycin (1.5 ng-20 ug). Ca⁺⁺ influx was monitored by fluorescence at 0.5-sec intervals over a 3-min time course.

FIG. 3. Substitution of the IgG CH3 domain with the C terminus of chicken surface/membrane μ chain yields antibodies that are expressed on the surface of DT40 cells, and form signaling-competent BCRs. The “chickenized” H and the original human L chains, along with GFP or a resistance gene, are expressed and translated through use of two promotors and an IRES sequence, in AID^{-/-/IgH}⁻/IgL⁻ DT40 cells (Reiser et al. 2000. J Virol 74: 10589-99). AID^{-/-}/IgH⁻/IgL⁻ DT40 cells are transfected, and cells expressing GFP or a selection marker are isolated by FACS or selection under zeocin. (A) Vector for Ab H and L chain expression in DT40 cells. “LTR”, long terminal repeat; “ch/hu Ig-H”, modified heavy chain of each antibody with the chicken C terminus of mIgM; “IRES”, internal ribosome entry site; “marker”, GFP or the zeocin selection marker; “CMV”, cytomegalovirus promoter; and “ori-Ig-L”, original human light chain of each Ab. (B) DT40 cells were analyzed for surface expression of chickenized b12 antibody 48 h post-transfection. Control unstained cells (I). Cells stained only with secondary anti-goat antibody (II). Untransfected cells stained with goat anti-human kappa light-chain antibody and anti-goat secondary (III). Cells transfected with expression vector encoding chickenized b12 IgH and kappa light chain (IV). Dashed ovals delineate background levels of anti-kappa light chain staining and solid ovals define cells expressing surface bound human-immunoglobulin. (C) Quantitative comparison between control (unstained, secondary only, and untransfected) and chickenized b12 transfected DT40 cells.

### 5. DETAILED DESCRIPTION OF THE INVENTION

Practice of the instant invention comprises introducing heterologous expression of immunoglobulin heavy and light chains of an antibody capable of neutralizing a targeted virus into cells capable of transmitting down-stream signals following B cell receptor activation, whereby the heavy chain of the neutralizing antibody anchors the immunoglobulin complex in the cellular plasma membrane and comprises any other amino acid sequences, domains, and/or post-translational modifications for B cell receptor signaling complex assembly and function. Such antibodies are either isolated from infected patients, and assayed as described above, or are isolated from sera of animals or humans immunized with one or more antigen of the present invention (see below). Cells used are either capable of transmitting down-stream signals following B cell receptor activation, or such signaling capacity is provided by co-expressing elements of the signaling molecules otherwise not present. In one particular embodiment, expression of endogenous immunoglobulin is eliminated or reduced. The cells are then assayed for signaling downstream of B cell receptor activation by cellular differentiation and/or proliferation-, biochemistry-, or molecular biology-based assays. In one particular embodiment, cells of the assay carry a reporter gene, such as, for example, but not limited to, green florescent protein, under the control of a promoter comprising an NFkappaB-responsive element, as NFkappaB activity is up-regulated following BCR activation.

### Cells that can be Used for the Assay

Any cell that is capable, or that is made to be capable of transmitting down-stream signals following B cell receptor activation, whether expressed endogenously or in trans, can be used for practice of the instant invention. These include, for example, but not limited to, mature B cells isolated from humans, vertebrates, mammals, birds, rodents, or other animals, transgenic animals, or immortalized cell lines. B cells can be immortalized by any method known in the art, preferably in such a way that does not interfere, or negatively affect the signal to noise ratio of the assay of the present invention (see, for example, Wiesner et al., 2008. Conditional immortalization of human B cells by CD40 ligation. PLoS ONE 3(1):e1464; Kusam & Dent, 2007. Common Mechanisms for the Regulation of B Cell Differentiation and Transformation by the Transcriptional Repressor Protein Bcl-6. Immunol Res 37(3):177-86).

A cell can be made to be capable of transmitting down-stream signals following B cell receptor activation by expressing other proteins of the B cell receptor signaling complex, including, for example, but not limited to, immunoglobulin alpha and beta, and any other molecules required for downstream signaling, including, for example, but not limited to, CD19, CD2, CD40, CD45, PIR-B, Fc□RIIB1, CRAC Channel (Ca⁺⁺), Lyn, Syk, Btk, PI₃K p85 & p110, Akt, PRK₂, PKC, TAK1, MEKKs, MKK3/46, MKK4/7, p38, JNK, JNK1/2, c-Raf, MEK1/2, Erk1/2, IKK, GSK-3, mTOR, p70 S6K, CaMK, IP₃R, SHP1, SHP2, SHIP, PTEN, Calcineurin, Rho, Rac/cdc42, RhoA, Rap, Ras, Rheb, Gab, BCAP, Shc, Dok-3, ezrin, BAM-32, clathrin, Nck, BLNK, Cbl, GRB2, LAB, STIM1, TSC2, CARMA1, Bcl10, CaM, IκB, RapL, Riam, PLC□2, MALT1, Vav, SOS, RasGRP, RasGAP, NFκB, NFAT, CREB, ATF02, Jun, Bcl-6, Egr-1, Elk-1, Bfl-1, Oct-2, Ets-1, FoxO, and Bcl-xL.

In a preferred embodiment, mature B cells from non-transgenic vertebrate animals, such as, for example, but not limited to, humans, mammals, birds, primates, or rodents are isolated by any means known to one of ordinary skill in the art and altered by any method known to one of ordinary skill in the art (see below) to suppress the expression of endogenous immunoglobulin. In another preferred embodiment, mature primary B cells are isolated by any means known to one of ordinary skill in the art from transgenic vertebrate animals, such as, for example, but not limited to, transgenic mammals, birds, primates, or rodents, in which gene expression is altered by any method known to one of ordinary skill in the art (see below) to suppress the expression of endogenous immunoglobulin. In another preferred embodiment, immortalized mature B cells from any vertebrates, such as, for example, but not limited to, humans, mammals, birds, primates, or rodents are altered by any method known to one of ordinary skill in the art (see below) to suppress the expression of endogenous immunoglobulin. In another preferred embodiment, the primary cells described above are immortalized by such methods as described above. In another preferred embodiment, commercially available, immortalized cell lines are used.

### The DT 40 and Other Cell Lines

A non-limiting example of a cell line that may be used to practice the invention described herein is the chicken DT 40 cell line. B-cell development in chicken and mammals is a very similar process, and the similarities are even greater at the molecular level and at the level of regulatory networks. The DT40 cell line is an avian leucosis virus-induced bursal B-cell lymphoma line that overexpresses c-myc and lacks p53 expression but otherwise has a stable pheno- and karyotype. The cell line appears to be arrested at the bursal stem cell stage of differentiation as it has on-going Ig diversification, and BCR ligation leads to apoptosis rather than proliferation. DT40 cells have several orders of magnitude higher homologous integration frequency than any other vertebrate cell lines described to date. This provides versatile options for deleting and replacing genes, which may prove very useful in refinement of an assay developed according to the methods of the instant invention (Kohonen P et al. 2007. Scand J Immunol 66:113-21).

DT40 cells express surface IgM, and have been used extensively to study BCR signaling. Much of what is known about the mechanisms of activation, interactions and hierarchy relationships of the multiple components that make up the BCR signaling pathway has been elucidated in DT40 cells (Winding & Berchtold, 2001. J Immunol Methods 249: 1-16; Kurosaki, 2002. Nat Rev Immunol 2:354-63). The DT40 cell line has also played a central role in elucidating the role and mechanism of action of AID, and has on-going Ig diversification due to AID expression (Winding & Berchtold, 2001. J Immunol Methods 249: 1-16; Arakawa, Saribasak, & Buerstedde, 2004. PLoS Biol 2:e179). For the purpose of expressing characterized immunoglobulin on the surface of these cells, however, somatic mutation would obscure the understanding of antibody/epitope-specific BCR signaling. Therefore an AID knock-out DT40 cell line is preferable (Arakawa, Hauschild, & Buerstedde, 2002. Science 295:1301-6).

Background would not be expected to be high due to cross-reactivity of DT40 endogenous surface immunoglobulin. However, heterologous co-expression of the antibodies selected for expression as BCR components may result in competition for other components of the BCR complex, such as the chicken Igα and lgβ chains. Therefore, a DT40 cell line that, in addition to its AID^{-/-} karyotype, lacks IgM heavy and light expression would be even more preferable (Arakawa, Hauschild, & Buerstedde, 2002. Science 295:1301-6).

Other cell lines that may be used to practice the invention include, as non-limiting examples, Ramos and CH12 cells. The Ramos human burkitts lymphoma cell line, like DT40 cells, is transformed by c-myc over-expression and does not possess the Epstein Barr Virus (EBV) genome. The cells have B lymphocyte characteristics, with surface associated μ and κ chains, and have been used extensively as model B lymphocytes for apoptosis studies. CH12 is a murine B-cell lymphoma-derived cell line that expresses both I-A and I-E class II molecules and μ/κ surface IgM with specificity for SRBC. CH12 cells resemble normal resting B cells in that they require both specific antigen and Ia-restricted T-cell help to induce their differentiation into antibody-secreting cells. CH12 cells can also be stimulated with LPS.

### Epitopes and Antigens

By “epitope” is intended the part of an antigenic molecule to which an antibody is produced and to which the antibody will bind. The term “epitope,” as used herein, refers to (a) portion(s) of a polypeptide having antigenic or immunogenic activity in an animal, preferably a vertebrate, more preferably a mammal, and most preferably in a human or a transgenic animal expressing relevant components of the human immune system. In a preferred embodiment, the present invention encompasses a polypeptide comprising an epitope, as well as the polynucleotide encoding this polypeptide. An “immunogenic epitope,” as used herein, is defined as a portion of a protein that elicits an antibody response in an animal or in human, as determined by any method known in the art, for example, by the methods for generating antibodies described below. (See, for example, Geysen H M et al. 1984. Proc Natl Acad Sci USA. 81:3998-4002). The term “antigenic epitope,” as used herein, is defined as a portion of a protein to which an antibody binds via its antigen binding region (i.e., domains containing the complementarity determining regions or antigen binding site) as determined by any method well known in the art, for example, by the antigenic assays described herein. Protein epitopes can comprise linear sequences of amino acid residues (i.e., residues within the epitope are arranged sequentially one after another in a linear fashion), nonlinear amino acid residues (referred to herein as “nonlinear epitopes”; residues of these epitopes are not arranged sequentially in an antigenic polypeptide), or both linear and nonlinear amino acid residues. Epitopes may also be conformational (i.e., comprised of one or more amino acid residues that are not contiguous in the primary structure of the protein but that are brought together by the secondary, tertiary or quaternary structure of a protein). The term “antigen epitope” as used herein refers to a three dimensional molecular structure (linear, non-linear, and/or conformational) that is capable of immunoreactivity with a monoclonal antibody. Antigen epitopes may comprise proteins, protein fragments, peptides, carbohydrates, lipids, oligopeptide mimics (i e, organic compounds that mimic the antibody binding properties of the antigen), and other molecules, or combinations thereof. Suitable oligopeptide mimics are described, inter alia, in PCT application U.S. 91/04282. Immunospecific binding excludes non-specific binding but does not exclude cross-reactivity with other antigens. Antigenic epitopes need not necessarily be immunogenic.

In the present invention, antigenic epitopes preferably contain a sequence of at least 3, at least 4, at least 5, at least 6, at least 7, at least 8, at least 9, at least 10, at least 11, at least 12, at least 13, at least 14, at least 15, at least 20, at least 25, at least 30, at least 40, at least 50 amino acids. Additional non-exclusive preferred antigenic epitopes include the antigenic epitopes disclosed herein, as well as portions thereof. Antigenic epitopes are useful, for example, to immunize patients against pathogenic viruses, or to raise antibodies, including monoclonal antibodies that specifically bind the epitope. Antigenic epitopes can be used as the target molecules in immunoassays. (See, for instance, Wilson I A et al. 1984. Cell. 37:767-778; Sutcliffe J G et al. 1983. Science 219(4585):660-666).

Furthermore, epitope bearing polypeptides of the invention may be modified, for example, by the addition of amino acids to the polypeptides, for example, but not limited to, at the amino- and/or carboxy-termini of the peptide. Such modifications may be performed, for example, to alter the conformation of the epitope bearing polypeptide such that the epitope will have a conformation more closely related to the structure of the epitope in the native protein. An example of a modified epitope-bearing polypeptide of the invention is a polypeptide in which one or more cysteine residues have been added to the polypeptide to allow for the formation of a disulfide bond between two cysteines, resulting in a stable loop structure of the epitope bearing polypeptide under non-reducing conditions. Disulfide bonds may form between a cysteine residue added to the polypeptide and a cysteine residue of the naturally occurring epitope, or may form between two cysteines which have both been added to the naturally occurring epitope bearing polypeptide. Additionally, it is possible to modify one or more amino acid residues of the naturally occurring epitope bearing polypeptide by substituting them with cysteines to promote the formation of disulfide bonded loop structures. Cyclic thioether molecules of synthetic peptides may be routinely generated using techniques known in the art and are described in PCT publication WO 97/46251, incorporated in its entirety by reference herein. Other modifications of epitope-bearing polypeptides contemplated by this invention include biotinylation.

Similarly, immunogenic epitopes can be used, for example, to activate BCRs of the invention, or to induce antibodies according to methods well known in the art. (See, for example, Wilson I A et al. 1984. Cell. 37:767-778; Sutcliffe J G et al. 1983. Science 219(4585):660-666; Bittle et al. 1985. J Gen Virol. 66:2347-2354, and Francis M J et al. 1985. J Gen Virol. 66:2347-2354; Chow M et al. 1985. Proc Natl Acad Sci USA. 82:910-914). The polypeptides comprising one or more immunogenic epitopes may be presented for eliciting an antibody response together with a carrier protein, such as an albumin, to humans or to an animal system (such as rabbit or mouse), or, if the polypeptide is of sufficient length (about 25 amino acids), the polypeptide may be presented without a carrier. However, immunogenic epitopes comprising as few as 8 to 10 amino acids have been shown to be sufficient to raise antibodies capable of binding to epitopes.

Antigen peptide may be coupled to a macromolecular carrier, such as, for example, but not limited to, keyhole limpet hemacyanin (KLH) or tetanus toxoid. For instance, peptides containing cysteine residues, and that are expressed or synthesized to contain cystein, for example, but not limited to, at the N- and C-termini, may be coupled to a carrier using a linker such as, but not limited to, maleimidobenzoyl-N-hydroxysuccinimide ester (MBS), while other peptides may be coupled to carriers using a more general linking agent such as glutaraldehyde.

Epitope bearing peptides of the invention may also be synthesized as multiple antigen peptides (MAPs) with or without T cell epitopes, first described by Tam JP, 1988 (Tam JP, 1988. Synthetic peptide vaccine design: synthesis and properties of a high-density multiple antigenic peptide system. Proc Natl Acad Sci USA. 85:5409 which is incorporated by reference herein in its entirety. MAPs consist of multiple copies of a specific peptide attached to a non-immunogenic lysine core. Map peptides usually contain four or eight copies of the peptide often referred to as MAP-4 or MAP-8 peptides. By way of non-limiting example, MAPs may be synthesized onto a lysine core matrix attached to a polyethylene glycol-polystyrene (PEG-PS) support. The peptide of interest is synthesized onto the lysine residues using 9-fluorenylmethoxycarbonyl (Fmoc) chemistry. For example, MAP resins, such as, for example, the Fmoc Resin 4 Branch and the Fmoc Resin 8 Branch which can be used to synthesize MAPs, are commercially available. Cleavage of MAPs from the resin may be performed with standard trifloroacetic acid (TFA)-based cocktails known in the art. Purification of MAPs, except for desalting, may not be not necessary. MAP peptides may be used as an immunizing vaccine which elicits antibodies that recognize both the MAP and the native protein from which the peptide was derived.

An immunogenic or antigenic epitope may also be fused to other polypeptide sequences. For example, the polypeptides of the present invention may be fused with the constant domain of immunoglobulins (IgA, IgE, IgG, IgM), or portions thereof (CH1, CH2, CH3, or any combination thereof and portions thereof) or, as non-limiting examples, albumin and transferin (including but not limited to recombinant human albumin or fragments or variants thereof, see, e.g., U.S. Pat. No. 5,876,969; EP Patent 0 413 622; U.S. Pat. No. 5,766,883; and U.S. Pat. No. 7,176,278), resulting in chimeric polypeptides. Such fusion proteins may facilitate purification and may increase half-life in vivo. This has been shown for chimeric proteins consisting of the first two domains of the human CD4-polypeptide and various domains of the constant regions of the heavy or light chains of mammalian immunoglobulins (see, for example, EP 394,827; Traunecker A et al. 1988. Nature. 331(6151):84-86). Enhanced delivery of an antigen across the epithelial barrier to the immune system has been demonstrated for antigens (e.g., insulin) conjugated to an FcRn binding partner such as IgG or Fc fragments (see, for example, PCT Publications WO 96/22024 and WO 99/04813). IgG Fusion proteins that have a disulfide-linked dimeric structure due to the IgG portion disulfide bonds have also been found to be more efficient in binding and neutralizing other molecules than monomeric polypeptides or fragments thereof alone (see, for example, Fountoulakis et al. 1995. J Biol. Chem. 270:3958-3964). Nucleic acids encoding the above epitopes can also be recombined with a gene of interest as an epitope tag (e.g., the hemagglutinin (“HA”) tag or flag tag) to aid in detection and purification of the expressed polypeptide. For example, a system described by Janknecht et al. allows for the ready purification of non-denatured fusion proteins expressed in human cell lines (Janknecht R et al. 1991. Proc Natl Acad Sci USA. 88(20):8972-8976).

Antigens may also be derivatives in that they are modified, i.e., by the covalent attachment of any type of molecule to the antigen. For example, but not by way of limitation, the antibody derivatives include antibodies that have been modified, e.g., by glycosylation, acetylation, pegylation, phosphylation, phosphorylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

Any of numerous methods of cleavage may be applied, including cleavage by cyanogen bromide, trypsin, chymotrypsin, papain, V8 protease, NaBH.sub.4, acetylation, formylation, oxidation, reduction, metabolic synthesis in the presence of tunicamycin, etc.

In addition, antigenic molecules of the invention may be chemically synthesized. For example, a peptide corresponding to a portion of a protein can be synthesized by use of a peptide synthesizer. Furthermore, if desired, non-classical amino acids or chemical amino acid analogs can be introduced as substitutions and/or additions into the sequence of one, any, both, several or all of the polypeptides of the complex.

Non-classical amino acids include, but are not limited to, the D-isomers of the common amino acids, fluoro-amino acids, designer amino acids such as beta-methyl amino acids, C gamma-methyl amino acids, N gamma-methyl amino acids, and amino acid analogs in general.

Examples of non-classical amino acids include: alpha-aminocaprylic acid, Acpa; (S)-2-aminoethyl-L-cysteine HCl, Aecys; aminophenylacetate, Afa; 6-amino hexanoic acid, Ahx; gamma-amino isobutyric acid and alpha-aminoisobytyric acid, Aiba; alloisoleucine, Aile; L-allylglycine, Alg; 2-amino butyric acid, 4-aminobutyric acid, and alpha-aminobutyric acid, Aba; p-aminophenylalanine, Aphe; b-alanine, Bal; p-bromophenylalaine, Brphe; cyclohexylalanine, Cha; citrulline, Cit; beta-chloroalanine, Clala; cycloleucine, Cle; p-cholorphenylalanine, Clphe; cysteic acid, Cya; 2,4-diaminobutyric acid, Dab; 3-amino propionic acid and 2,3-diaminopropionic acid, Dap; 3,4-dehydroproline, Dhp; 3,4-dihydroxylphenylalanine, Dhphe; p-fluorophenylalanine, Fphe; D-glucoseaminic acid, Gaa; homoarginine, Hag; delta-hydroxylysine HCl, Hlys; DL-beta-hydroxynorvaline, Hnyl; homoglutamine, Hog; homophenylalanine, Hoph; homoserine, Hos; hydroxyproline, Hpr; p-iodophenylalanine, Iphe; isoserine, Ise; alpha-methylleucine, Mle; DL-methionine-5-methylsulfoniumchloide, Msmet; 3-(1-naphthyl)alanine, 1Nala; 3-(2-naphthyl)alanine, 2Nala; norleucine, Nle; N-methylalanine, Nmala; Norvaline, Nva; O-benzylserine, Obser; O-benzyltyrosine, Obtyr; O-ethyltyrosine, Oetyr; O-methylserine, Omser; O-methylthreonine, Omthr; O-methyltyrosine, Omtyr; Ornithine, Orn; phenylglycine; penicillamine, Pen; pyroglutamic acid, Pga; pipecolic acid, Pip; sarcosine, Sar; t-butylglycine; t-butylalanine; 3,3,3-trifluoroalanine, Tfa; 6-hydroxydopa, Thphe; L-vinylglycine, Vig; (−)-(2R)-2-amino-3-(2-aminoethylsulfonyl)propanoic acid dihydroxochloride, Aaspa; (2S)-2-amino-9-hydroxy-4,7-dioxanonanoic acid, Ahdna; (2S)-2-amino-6-hydroxy-4-oxahexanoic acid, Ahoha; (−)-(2R)-2-amino-3-(2-hydroxyethylsulfonyl)propanoic acid, Ahsopa; (−)-(2R)-2-amino-3-(2-hydroxyethylsulfanyl)propanoic acid, Ahspa; (2S)-2-amino-12-hydroxy-4,7,10-trioxadodecanoic acid, Ahtda; (2S)-2,9-diamino-4,7-dioxanonanoic acid, Dadna; (2S)-2,12-diamino-4,7,10-trioxadodecanoic acid, Datda; (S)-5,5-difluoronorleucine, Dfnl; (S)-4,4-difluoronorvaline, Dfnv; (3R)-1-1-dioxo-[1,4]thiaziane-3-carboxylic acid, Dtca; (S)-4,4,5,5,6,6,6-heptafluoronorleucine, Hfnl; (S)-5,5,6,6,6-pentafluoronorleucine, Pfnl; (S)-4,4,5,5,5-pentafluoronorvaline, Pfnv; and (3R)-1,4-thiazinane-3-carboxylic acid, Tca. Furthermore, the amino acid can be D (dextrorotary) or L (levorotary). For a review of classical and non-classical amino acids, see Sandberg et al. (Sandberg M et al. 1998. J Med Chem. 41(14): 2481-91).

### Antigens and Epitopes of the Present Invention

Antibodies of the present invention are generated, by non-limiting example, by immunizing animals with polypeptides comprising, or alternatively consisting of, at least one epitope of the invention. An epitope of the invention is an epitope as described above, is a viral protein, or any mutants, fragments, variants, derivatives, conjugates, multimers, or fusions thereof. Alternatively, the epitope is a non-viral polypeptide or peptidomimetic that structurally and/or antigenically mimics the epitope of the viral protein described above, whereby an antibody that specifically interact with the epitope crossreacts with an epitope of the viral protein.

Antibodies that bind the epitopes of the present invention neutralize or broadly neutralize the targeted virus, as described herein under antigenicity assays, immunogenicity assays, neutralization assays, and viral uptake assays.

### Production of Antigen

Polypeptides, mutants, fragments, variants, derivates, multimers, conjugates, and fusion proteins of the above sequences, which function as epitopes, may be synthesized or produced by any conventional means. (See, e.g., Houghten R A. 1985. Proc Natl Acad Sci USA. 82(15):5131-5135, further described in U.S. Pat. No. 4,631,211, both incorporated in their entirety by reference herein). In one embodiment of the invention, the antigen is expressed recombinantly from a nucleotide sequence encoding the amino acid sequence of a polypeptide antigen in prokaryotic or eukaryotic expression systems, such as, for example, but not limited to, E. coli, yeast, insect, such as, for example Sf9 cells infected by an antigen-specific baculovirus (expression vector) or drosophila cell lines, murine, such as, for example, Chinese Hamster Ovary (CHO) cells, simian, such as, for example, COS cells, human cells lines, such as, for example, HeLa or HEP293 cells, or any other system for recombinant production of protein. Mutations may be introduced into the DNA encoding the polypeptides may be introduced by any methods known to one of ordinary skill in the art.

For example, epitope bearing polypeptides of the invention may be expressed in baculovirus infected insect cells, such as Sf9 cells, whereby such cells may be used as the immunogen. Production of the Sf 9 (Spodoptera frugiperda) cells is disclosed in U.S. Pat. No. 6,004,552, incorporated herein by reference. Briefly, sequences encoding the epitope bearing peptide or protein are recombined into a baculovirus using transfer vectors. The plasmids are co-transfected with wild-type baculovirus DNA into Sf 9 cells. Recombinant baculovirus-infected Sf 9 cells expressing the desired epitope bearing polypeptide are identified by standard methods known to one of ordinary skill in the art, and clonally purified.

### Immunization Methods

Epitope-bearing polypeptides of the present invention may be used to induce antibodies according to methods well known in the art including, but not limited to, in vivo immunization, in vitro immunization, and phage display methods. See, for example, Wilson I A et al. 1984. Cell. 37:767-778; Sutcliffe J G et al. 1983. Science 219(4585):660-666, and Francis M J et al. 1985. J Gen Virol. 66:2347-2354, and Bittle et al. 1985. J Gen Virol. 66:2347-2354, all of which are incorporated in their entirety by reference herein.

For in vivo immunizations, animals such as, for example, humans, rabbits, rats and mice are immunized with either free or carrier-coupled peptides or MAP peptides of emulsions containing an effective amount of peptide protein complex, or carrier protein, often an amount between 50 and 200 micro-g/injection is sufficient; the epitope bearing polypeptide, free or carrier-coupled, is preferably emulsified in Freund's adjuvant or any other adjuvant known for stimulating an immune response. Immunization can also be performed by mixing or emulsifying the antigen-containing solution in saline, preferably in an adjuvant such as Freund's complete adjuvant, and injecting the mixture or emulsion parenterally, generally subcutaneously, intramuscularly, intraperitoneally and/or intradermally, though other routes may be effective, as well. One or several booster injections of the above antigen, for example, but not limited to, in saline, and preferably using an adjuvant, such as, but not limited to, Freund's incomplete adjuvant, may be useful or needed, for instance, at intervals of effective periods of time, often about two weeks, to provide a useful titer of antibody which can be detected, for example, by ELISA assay using free polypeptide adsorbed to a solid surface.

Polyclonal antisera are obtained to determine the existence of neutralizing antibodies by bleeding the immunized animal by any method known to one of ordinary skill in the art. For example, the animals are bled into a glass or plastic container, incubating the blood at 25 degrees C. for one hour, followed by incubating at 4 degrees C. for 2-18 hours. The serum is recovered by centrifugation (e.g., 1,000.times.g for 10 minutes). About 20-50 ml per bleed may be obtained from rabbits. The titer of antibodies in serum from an immunized animal may be increased by selection of antigen-specific antibodies, for instance, by adsorption to the peptide on a solid support and elution of the selected antibodies according to methods well known to a person of ordinary skill in the art.

One may alternatively generate antibodies by in vitro immunization using methods known in the art, preferably for the production of monoclonal antibodies, which for the purposes of this invention is considered equivalent to in vivo immunization.

### Immunogenic Analysis

Immunogenic analyses, i.e. the determination of whether an antigen of the present invention, and any derivates, analogs, orthologs, homologs, fragments, chimers, or fusion proteins thereof, and one, any, both, several or all of the polypeptides of a complex, and any derivates, analogs, orthologs, homologs, fragments, chimers, or fusion proteins thereof, identified as immunogens for use in vaccines, and/or used as immunogens to generate antibodies that bind to antigenic structures and neutralize one or more targeted viruses, may be performed by any method known in the art. Such methods include, as nonlimiting examples, those described in detail by Dey et al. 2007 (Dey et al., 2007. Characterization of Human Immunodeficiency Virus Type 1 Monomeric and Trimeric gp120 Glycoproteins Stabilized in the CD4-Bound State: Antigenicity, Biophysics, and Immunogenicity. J Virol 81(11): 5579-5593) and Beddows et al., 2006 (Beddows et al., 2007. A comparative immunogenicity study in rabbits of disulfidestabilized proteolytically cleaved, soluble trimeric human immunodeficiency virus type 1 gp140, trimeric cleavage-defective gp140 and momomeric gp120. Virol 360: 329-340).

### Methods of Isolating Cells Producing Polypeptide Antigens

Any methods known to one of ordinary skill in the art may be used to identify and/or isolated cells expressing antibodies of the present invention. For example, after immunization of the animal, the spleen (and optionally, several large lymph nodes) are removed and dissociated into single cells. The spleen cells may be screened by applying a cell suspension to a plate or well coated with the antigen of interest. The B cells expressing membrane bound immunoglobulin specific for the antigen bind to the plate and are not rinsed away. Resulting B cells, or all dissociated spleen cells, are then induced to fuse with myeloma cells to form hybridomas, and are cultured in a selective medium. The resulting cells are plated by serial dilution and are assayed for the production of antibodies that specifically bind the antigen and epitope of interest (and that do not bind to unrelated antigens, see below), or that functionally neutralize viral infection, as determined, for example, by neutralization assays described herein. The selected monoclonal antibody (mAb)-secreting hybridomas are then cultured either in vitro (e.g., in tissue culture bottles or hollow fiber reactors), or in vivo (as ascites in mice).

Antibodies or antibody fragments can also be isolated from antibody phage libraries generated using the techniques described in, for example, McCafferty et al. 1990. Nature 348: 552-554; and U.S. Pat. No. 5,514,548; Clackson et al. 1991. Nature 352: 624-628; and Marks et al. 1991. J Mol Biol. 222: 581-597 describe the isolation of murine and human antibodies, respectively, using phage libraries (all incorporated in their entirety by reference herein). Subsequent publications describe the production of high affinity (nM range) human antibodies by chain shuffling (Marks J D et al. 1992. Biotechnology. 10 (7):779-783, incorporated by reference herein), as well as combinatorial infection and in vivo recombination as a strategy for constructing very large phage libraries (Waterhouse P et al. 1993. Nucleic. Acids Res. 21(9):2265-2266, incorporated by reference herein).

For example, the antibodies of the present invention can also be generated using various phage display methods known in the art. In phage display methods, functional antibody domains are displayed on the surface of phage particles which carry the polynucleotide sequences encoding them. In a particular embodiment, such phage can be utilized to display antigen binding domains expressed from a repertoire or combinatorial antibody library (e.g., human or murine). Phage expressing an antigen binding domain that binds the antigen of interest can be selected or identified with antigen, e.g., using labeled antigen or antigen bound or captured to a solid surface or bead. Phage used in these methods are typically filamentous phage including fd and M13 binding domains expressed from phage with Fab, Fv or disulfide stabilized Fv antibody domains recombinantly fused to either the phage gene III or gene VIII protein. Thus, these techniques are viable alternatives to traditional monoclonal antibody hybridoma techniques for isolation of monoclonal antibodies.

Other examples of phage display methods that can be used to make the antibodies of the present invention include those disclosed in Brinkmann U et al. 1995. J Immunol Methods. 182(1): 41-50; Ames R S et al. 1995. J Immunol Methods 184(2): 177-186; Kettleborough C A et al. 1994. Eur J. Immunol. 24(4): 952-958; Persic L et al. 1997. Gene 187(1): 9-18; Burton D R & Barbas C F 3rd. 1994. Adv Immunol. 57: 191-280; PCT application No. PCT/GB91/01134; PCT publications WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108; each of which is incorporated herein by reference in its entirety.

Another well known method for producing monoclonal human B cell lines is transformation using Epstein Barr Virus (EBV). Protocols for generating EBV-transformed B cell lines are commonly known in the art, such as, for example, the protocol outlined in Chapter 7.22 of Current Protocols in Immunology, Coligan et al., Eds., 1994, John Wiley & Sons, NY, which is hereby incorporated in its entirety by reference herein. The source of B cells for transformation is commonly human peripheral blood, but B cells for transformation may also be derived from other sources including, but not limited to, lymph nodes, tonsil, spleen, tumor tissue, and infected tissues. Tissues are generally made into single cell suspensions prior to EBV transformation. Additionally, steps may be taken to either physically remove or inactivate T cells (e.g., by treatment with cyclosporin A) in B cell-containing samples, because T cells from individuals seropositive for anti-EBV antibodies can suppress B cell immortalization by EBV. In general, the sample containing human B cells is inoculated with EBV, and cultured for 3-4 weeks. A typical source of EBV is the culture supernatant of the B95-8 cell line (ATCC #VR-1492). Physical signs of EBV transformation can generally be seen towards the end of the 3-4 week culture period. By phase-contrast microscopy, transformed cells may appear large, clear, hairy and tend to aggregate in tight clusters of cells. Initially, EBV lines are generally polyclonal. However, over prolonged periods of cell cultures, EBV lines may become monoclonal as a result of the selective outgrowth of particular B cell clones. Alternatively, polyclonal EBV transformed lines may be subcloned (e.g., by limiting dilution culture) or fused with a suitable fusion partner and plated at limiting dilution to obtain monoclonal B cell lines. Suitable fusion partners for EBV transformed cell lines include mouse myeloma cell lines (e.g., SP2/0, X63-Ag8.653), heteromyeloma cell lines (human x mouse; e.g., SPAM-8, SBC-H20, and CB-F7), and human cell lines (e.g., GM 1500, SKO-007, RPMI 8226, and KR-4). Thus, the present invention also provides a method of generating polyclonal or monoclonal human antibodies against polypeptides of the invention or fragments thereof, comprising EBV-transformation of human B cells.

### Antibodies

The term “antibody,” as used herein, refers to immunoglobulin molecules and immunologically active portions or fragments of immunoglobulin molecules, including T cell receptor molecules, i.e., molecules that contain an antigen binding site that immunospecifically binds an antigen. As such the term “antibody” encompasses not only whole antibody molecules, but also antibody multimers and antibody fragments and/or variants (including derivatives) of antibodies, antibody multimers and antibody fragments. Examples of molecules which are described by the term “antibody” herein include, but are not limited to: single chain Fvs (scFvs), Fab fragments, Fab′ fragments, F(ab′)2, disulfide linked Fvs (sdFvs), Fvs, and fragments comprising or alternatively consisting of, either a VL or a VH domain. The term “single chain Fv” or “scFv” as used herein refers to a polypeptide comprising a VL domain of antibody linked to a VH domain of an antibody.

By “isolated antibody” is intended an antibody removed from its native environment. Thus, an antibody produced by, purified from and/or contained within a hybridoma and/or a recombinant host cell is considered isolated for purposes of the present invention.

The basic antibody structural unit is known to comprise a tetramer. Each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. Human light chains are classified as kappa and lambda light chains. Heavy chains are classified as mu, delta, gamma, alpha, or epsilon, and define the antibody's isotype as IgM, IgD, IgG, IgA, and IgE, respectively. See generally, Fundamental Immunology Ch. 7 (Paul W. ed. 1989. 2nd ed. Raven Press, N.Y.), incorporated by reference in its entirety for all purposes. The variable regions of each light/heavy chain pair form the antibody binding site. Thus, an intact IgG antibody has two binding sites. Except in bifunctional or bispecific antibodies, the two binding sites are the same.

The binding site chains all exhibit the same general structure of relatively conserved framework regions (FR) joined by three hyper variable regions, also called complementarity determining regions or CDRs. The CDRs of the heavy and the light chains of a pair are aligned by the framework regions, enabling binding to a specific epitope. From N-terminal to C-terminal, both light and heavy chains comprise the domains FR1, CDR1, FR2, CDR2, FR3, CDR3 and FR4. The assignment of amino acids to each domain is often in accordance with the definitions of Kabat Sequences of Proteins of Immunological Interest (National Institutes of Health, Bethesda, Md., 1987 and 1991), or Chothia C & Lesk A M 1987. J. Mol. Biol. 196(4):901-917; Chothia C et al. 1989. Nature 342(6252):877-883, incorporated by reference herein).

By “Fab” is intended a monovalent antigen-binding fragment of an immunoglobulin that is composed of the light chain and part of the heavy chain. By F(ab′).sub.2 is intended a bivalent antigen-binding fragment of an immunoglobulin that contains both light chains and part of both heavy chains. By “single-chain Fv” or “sFv” antibody fragments is intended fragments comprising the V.sub.H and V.sub.L domains of an antibody, wherein these domains are present in a single polypeptide chain. See, for example, U.S. Pat. Nos. 4,946,778, 5,260,203, 5,455,030, and 5,856,456, herein incorporated by reference. Generally, the Fv polypeptide further comprises a polypeptide linker between the V_{H} and V_{L} domains that enables the sFv to form the desired structure for antigen-binding. For a review of sFv see Pluckthun (1994) in The Pharmacology of Monoclonal Antibodies, Vol. 113, ed. Rosenburg and Moore (Springer-Verlag, New York), pp. 269-315. The V.sub.H and V.sub.L domain complex of Fv fragments may also be stabilized by a disulfide bond (U.S. Pat. No. 5,747,654, incorporated by reference herein)

A bispecific or bifunctional antibody is a hybrid antibody having two different heavy/light chain pairs and two different binding sites. Bispecific antibodies can be produced by a variety of methods including fusion of hybridomas or linking of Fab′ fragments. See, for example, Songsivilai S & Lachmann P G. 1990. Clin Exp Immunol. 79(3):315-321, Kostelny S A et al. 1992. J Immunol. 148(5):1547-1553 (both incorporated by reference herein). In addition, bispecific antibodies may be formed as “diabodies” (Holliger P et al. 1993. Proc Natl Acad Sci USA. 90(14):6444-6448, incorporated by reference herein) or “janusins” (see Traunecker A et al. 1991. EMBO J 10(12):3655-3659; and Traunecker A et al. 1992. Int J Cancer Suppl. 7:51-52, both incorporated by reference herein).

Antibodies can be made to multimerize naturally or through recombinant DNA techniques. IgM and IgA naturally form antibody multimers through the interaction with the J chain polypeptide. Non-IgA or non-IgM molecules, such as IgG molecules, can be engineered to contain the J chain interaction domain of IgA or IgM, thereby conferring the ability to form higher order multimers on the non-IgA or non-IgM molecules. (see, for example, Chintalacharuvu K R et al. 2001. Clin Immunol. 101(1):21-31; and Frigerio L et al. 2000. Plant Physiology 123(2):1483-94, both of which are hereby incorporated by reference in their entireties.) ScFv dimers can also be formed through recombinant techniques known in the art; an example of the construction of scFv dimers is given in Goel A et al. 2000. Cancer Research. 60(24):6964-6971, which is hereby in its entirety incorporated by reference. Antibody multimers may be purified using any suitable method known in the art, including, but not limited to, size exclusion chromatography.

Specific binding or immunospecific binding by an antibody means that the antibody binds (a) specific antigen molecule(s), or fragments, variants, or derivates, multimers, or fusion proteins thereof, but does not significantly bind to (i.e., cross react with) antigens, such as, for example, other structurally or functionally related proteins, or proteins with sequence homology. An antibody that binds the antigen of this invention and does not cross-react with other proteins is not necessarily an antibody that does not bind said other proteins under any or all conditions; rather, the antigen-specific antibody of the invention preferentially binds the antigen compared to its ability to bind said other antigens such that it will be suitable for use in at least one type of treatment, i.e. result in no unreasonable adverse effects in treatment.

Given that antigen-specific antibodies bind to epitopes of the antigen, an antibody that specifically binds antigen may or may not bind fragments of the antigen and/or variants of the antigen (e.g., proteins that are at least 95% identical to the antigen) depending on the presence or absence of the epitope bound by a given antigen-specific antibody in the antigen fragment or variant. Likewise, antigen-specific antibodies of the invention may bind species orthologues of the antigen (including fragments thereof) depending on the presence or absence of the epitope recognized by the antibody in the orthologue. Additionally, antigen-specific antibodies of the invention may bind modified forms of the antigen, for example, antigen fusion proteins. In such a case when antibodies of the invention bind the antigen fusion proteins, the antibody must make binding contact with the antigen moiety of the fusion protein in order for the binding to be specific for the antigen. Antibodies that specifically bind the antigen can be identified, for example, by immunoassays or other techniques known to those of skill in the art, e.g., the immunoassays described below.

### Antibodies of the Invention

The present invention also provides antibodies that are generated by immunization with an antigen identified as broadly neutralizing by the methods of this invention (see below), and immunospecifically bind to a viral antigen of the invention (see above), or to a polypeptide or a mutant, fragment, variant, derivative, or fusion protein thereof, and thereby neutralize the virus. Membrane-bound forms of the antibodies generated by the methods of this can also be expressed in cells such that they form a signaling competent B cell receptor complex; this allows for an iterative process, by which antibodies are used to identify antigen, antigen is used to generate antibodies, which in turn, are again used to identify antigen, etc.

Immunospecific binding is determined by immunoassays well known to one of ordinary skill in the art for assaying specific antibody-antigen interactions (see below). Immunospecific binding of an antibody is binding of said antibody with a K_{d} at least one half of an order of magnitude, preferably two, more preferably three, even more preferably four or more orders of magnitude lower that the Kd of its binding to the same antigen not engineered according to the methods of the present invention.

Included in the invention are neutralizing antibodies which bind the viral epitope, and competitively prevent binding of the virus to the host cell virus receptor, as well as antibodies which bind the virus and induce a conformational chance in the viral envelope proteins, and thereby inhibit binding of the virus to the host cell receptor, or thereby inhibit conformational changes of the viral protein that are required for viral binding to the host cell receptor, fusion of the viral membrane with the host cell membrane, uptake of viral genomic material (i.e. nucleic acids), or for any other process or processes required for a productive infection of the host cell, thereby neutralizing the virus.

Antibodies of the invention include, but are not limited to, monoclonal, multispecific, human, humanized or chimeric antibodies, and anti-idiotypic (anti-Id) antibodies (including, e.g., anti-Id antibodies to antibodies against a receptor molecule of the antigen of the present invention). The immunoglobulin molecules of the invention can be of any type (e.g., IgG, IgE, IgM, IgD, IgA and IgY), class (e.g., IgG1, IgG2, IgG3, IgG4, IgA1and IgA2) or subclass of immunoglobulin molecule. In a preferred embodiment, the immunoglobulin is an IgM isotype. In a preferred embodiment, the immunoglobulin is an IgD isotype. In a preferred embodiment, the immunoglobulin is an IgG1 isotype. In another preferred embodiment, the immunoglobulin is an IgG2 isotype. In another preferred embodiment, the immunoglobulin is an IgG4 isotype. Immunoglobulins may have both a heavy and light chain. An array of IgG, IgE, IgM, IgD, IgA, and IgY heavy chains may be paired with a light chain of the kappa or lambda forms.

Preferably the antibodies of the present invention are human or humanized antibodies. The antibodies of the invention may be from any animal origin including birds and mammals. In another embodiment of the invention, the antibodies are murine (e.g., mouse and rat), donkey, sheep, rabbit, goat, guinea pig, camel, horse, or chicken. As used herein, “human” antibodies include antibodies having the amino acid sequence of a human immunoglobulin and include antibodies isolated from human immunoglobulin libraries or from animals transgenic for one or more human immunoglobulin and that do not express endogenous immunoglobulins, as described below and, for example in, U.S. Pat. No. 5,939,598 incorporated by reference herein in its entirety. Furthermore, human antibodies may be humanized, also as described in detail below.

Antibodies of the present invention may also be described or specified in terms of their cross-reactivity. Antibodies that do not bind any other analog, ortholog, or homolog of the antigen of the present invention are included. Antibodies that bind polypeptides with at least 95%, at least 90%, at least 85%, at least 80%, at least 75%, at least 70%, at least 65%, at least 60%, at least 55%, and at least 50% identity (as calculated using methods known in the art) to the antigen are also included in the present invention. In specific embodiments, antibodies of the present invention cross-react with viral homologs of the antigen polypeptide and the corresponding epitopes thereof. Antibodies that do not bind polypeptides with less than 95%, less than 90%, less than 85%, less than 80%, less than 75%, less than 70%, less than 65%, less than 60%, less than 55%, and less than 50% identity (as calculated using methods known in the art) to an antigen polypeptide of the present invention are also included in the present invention. In a specific embodiment, the above-described cross-reactivity is with respect to any single specific antigenic or immunogenic polypeptide, or combination(s) of 2, 3, 4, 5, or more of the specific antigenic and/or immunogenic polypeptides disclosed herein.

By way of non-limiting example, an antibody may be considered to bind a first antigen preferentially if it binds said first antigen with a dissociation constant (K.sub.D) that is less than the antibody's K.sub.D for the second antigen. In another non-limiting embodiment, an antibody may be considered to bind a first antigen preferentially if it binds said first antigen with an affinity that is at least one order of magnitude less than the antibody's K.sub.D for the second antigen. In another non-limiting embodiment, an antibody may be considered to bind a first antigen preferentially if it binds said first antigen with an affinity that is at least two orders of magnitude less than the antibody's K.sub.D for the second antigen.

In another non-limiting embodiment, an antibody may be considered to bind a first antigen preferentially if it binds said first antigen with an off rate (k.sub.off) that is less than the antibody's k.sub.off for the second antigen. In another non-limiting embodiment, an antibody may be considered to bind a first antigen preferentially if it binds said first antigen with an affinity that is at least one order of magnitude less than the antibody's k.sub.off for the second antigen. In another non-limiting embodiment, an antibody may be considered to bind a first antigen preferentially if it binds said first antigen with an affinity that is at least two orders of magnitude less than the antibody's k.sub.off for the second antigen.

Antibodies of the present invention may also be described or specified in terms of their binding affinity to the viral polypeptides, or fragments, variants, or derivatives thereof.

Preferred binding affinities include those with a dissociation constant or K.sub.D less than 5 times 10.sup.-2 M, 10.sup.-2 M, 5 times 10.sup.-3 M, 10.sup.-3 M, 5 times 10.sup.-4 M, 10.sup.-4 M. More preferred binding affinities include those with a dissociation constant or K.sub.D less than 5 times 10.sup.-5 M, 10.sup.-5 M, 5 times 10.sup.-6 M, 10.sup.-6 M, 5 times 10.sup.-7 M, 10.sup.-7 M, 5 times 10.sup.-8 M or 10.sup.-8 M. Even more preferred binding affinities include those with a dissociation constant or K.sup.D less than 5 times 10.sup.-9 M, 10.sup.-9 M, 5 times 10.sup.-10 M, 10.sup.-10 M, 5 times 10.sup.-11 M, 10.sup.-11 M, 5 times 10.sup.-12 M, 10.sup.-12 M, 5 times 10.sup.-13 M, 10.sup.-13 M, 5 times 10.sup.-14 M, 10.sup.-14 M, 5 times 10.sup.-15 M, or 10.sup.-15 M.

In specific embodiments, antibodies of the invention bind antigen polypeptides of the invention, or fragments or variant thereof with an off rate (k.sub.off) of less than or equal to 5 times 10.sup.-2 sec.sup.-1, 10.sup.-2 sec.sup.-1, 5 times 10.sup.-3 sec.sup.-1 or 10.sup.-3 sec.sup.-1. More preferably, antibodies of the invention bind antigen polypeptides of the invention with an off rate (k.sub.off) less than or equal to 5 times 10.sup.-4 sec.sup.-1, 10.sup.-4 sec.sup.-1, 5 times 10.sup.-5 sec.sup.-1, or 10.sup.-5 sec.sup.-1, 5 times 10.sup.-6 sec.sup.-1, 10.sup.-6 sec.sup.-1, 5 times 10.sup.-7 sec.sup.-1, or 10.sup.-7 sec.sup.-1.

In other embodiments, antibodies of the invention bind antigen polypeptides of the invention with an on rate (k.sub.on) of greater than or equal to 10.sup.3 M.sup.-1 sec.sup.-1, 5 times 10.sup.3 M.sup.-1 sec.sup.-1, 10.sup.4 M.sup.-1 sec.sup.-1 or 5 times 10.sup.4 M.sup.-1 sec.sup.-1. More preferably, antibodies of the invention bind antigen polypeptides of the invention with an on rate (k.sub.on) greater than or equal to 10.sup.5 M.sup.-1 sec.sup.-1, 5 times 10.sup.5 M.sup.-1 sec.sup.-1, 10.sup.6 M.sup.-1 sec.sup.-1, or 5 times 10.sup.6 M.sup.-1 sec.sup.-1, or 10.sup.7 M.sub.-1 sec.sub.-1.

In one embodiment of the present invention, antibodies that immunospecifically bind antigen polypeptides of the invention, comprise a polypeptide having the amino acid sequence of any one of the heavy chains expressed by a cell line expressing an antibody of the invention and/or any one of the light chains expressed by an a cell line expressing an antibody of the invention. In another embodiment of the present invention, antibodies that immunospecifically bind antigen polypeptides of the invention, comprise a polypeptide having the amino acid sequence of any one of the VH domains of a heavy chain expressed by a cell line expressing an antibody of the invention and/or any one of the VL domains of a light chain expressed by a cell line expressing an antibody of the invention. In preferred embodiments, antibodies of the present invention comprise the amino acid sequence of a VH domain and VL domain expressed a cell line expressing a single antibody of the invention. In alternative embodiments, antibodies of the present invention comprise the amino acid sequence of a VH domain and a VL domain expressed by two different cell lines expressing an antibody of the invention. Molecules comprising, or alternatively consisting of, antibody fragments or variants of the VH and/or VL domains expressed by a cell line expressing an antibody of the invention that immunospecifically bind the antigen of the invention are also encompassed by the invention, as are nucleic acid molecules encoding these VH and VL domains, molecules, fragments and/or variants.

The present invention also provides antibodies that immunospecifically bind antigen polypeptides of the invention, wherein said antibodies comprise, or alternatively consist of, a polypeptide having an amino acid sequence of any one, two, three, or more of the VH CDRs contained in a heavy chain expressed by one or more cell lines expressing an antibody of the invention. In particular, the invention provides antibodies that immunospecifically bind antigen polypeptides of the invention, comprising, or alternatively consisting of, a polypeptide having the amino acid sequence of a VH CDR1 contained in a heavy chain expressed by one more cell lines expressing an antibody of the invention. In another embodiment, antibodies that immunospecifically bind antigen polypeptides of the invention, comprise, or alternatively consist of, a polypeptide having the amino acid sequence of a VH CDR2 contained in a heavy chain expressed by one or more cell lines expressing an antibody of the invention. In a preferred embodiment, antibodies that immunospecifically bind antigen polypeptides of the invention, comprise, or alternatively consist of a polypeptide having the amino acid sequence of a VH CDR3 contained in a heavy chain expressed by one or more cell lines expressing an antibody of the invention. Molecules comprising, or alternatively consisting of, these antibodies, or antibody fragments or variants thereof, that immunospecifically bind antigen polypeptides of the invention are also encompassed by the invention, as are nucleic acid molecules encoding these antibodies, molecules, fragments and/or variants.

The present invention also provides antibodies that immunospecifically bind antigen polypeptides of the invention, wherein said antibodies comprise, or alternatively consist of, a polypeptide having an amino acid sequence of any one, two, three, or more of the VL CDRs contained in a light chain expressed by one or more cell lines expressing an antibody of the invention. In particular, the invention provides antibodies that immunospecifically bind antigen polypeptides of the invention, comprising, or alternatively consisting of, a polypeptide having the amino acid sequence of a VL CDR1 contained in a light chain expressed by one or more cell lines expressing an antibody of the invention. In another embodiment, antibodies that immunospecifically bind antigen polypeptides of the invention, comprise, or alternatively consist of, a polypeptide having the amino acid sequence of a VL CDR2 contained in a light chain expressed by one or more cell lines expressing an antibody of the invention. In a preferred embodiment, antibodies that immunospecifically bind antigen polypeptides of the invention, comprise, or alternatively consist of a polypeptide having the amino acid sequence of a VL CDR3 contained in a light chain expressed by one or more cell line expressing an antibody of the invention. Molecules comprising, or alternatively consisting of, these antibodies, or antibody fragments or variants thereof, that immunospecifically bind antigen polypeptides of the invention are also encompassed by the invention, as are nucleic acid molecules encoding these antibodies, molecules, fragments and/or variants.

The present invention also provides antibodies (including molecules comprising, or alternatively consisting of, antibody fragments or variants) that immunospecifically bind antigen polypeptides of the invention, wherein said antibodies comprise, or alternatively consist of, one, two, three, or more VH CDRs and one, two, three or more VL CDRs, as contained in a heavy chain or light chain expressed by one or more cell lines expressing an antibody of the invention. In particular, the invention provides for antibodies that immunospecifically bind antigen polypeptides of the invention, wherein said antibodies comprise, or alternatively consist of, a VH CDR1 and a VL CDR1, a VH CDR1 and a VL CDR2, a VH CDR1 and a VL CDR3, a VH CDR2 and a VL CDR1, VH CDR2 and VL CDR2, a VH CDR2 and a VL CDR3, a VH CDR3 and a VH CDR1, a VH CDR3 and a VL CDR2, a VH CDR3 and a VL CDR3, or any combination thereof, of the VH CDRs and VL CDRs contained in a light chain or light chain expressed by one or more cell lines expressing an antibody of the invention. In a preferred embodiment, one or more of these combinations are from a single antibody expressing cell line of the invention. Molecules comprising, or alternatively consisting of, fragments or variants of these antibodies, that immunospecifically bind antigen polypeptides of the invention are also encompassed by the invention, as are nucleic acid molecules encoding these antibodies, molecules, fragments or variants.

The present invention also provides for nucleic acid molecules, generally isolated, encoding an antibody of the invention (including molecules comprising, or alternatively consisting of, antibody fragments or variants thereof). In a specific embodiment, a nucleic acid molecule of the invention encodes an antibody (including molecules comprising, or alternatively consisting of, antibody fragments or variants thereof), comprising, or alternatively consisting of, a VH domain having an amino acid sequence of any one of the VH domains of a heavy chain expressed by a cell line expressing an antibody of the invention and a VL domain having an amino acid sequence of a light chain expressed by a cell line expressing an antibody of the invention. In another embodiment, a nucleic acid molecule of the invention encodes an antibody (including molecules comprising, or alternatively consisting of, antibody fragments or variants thereof), comprising, or alternatively consisting of, a VH domain having an amino acid sequence of any one of the VH domains of a heavy chain expressed by a cell line expressing an antibody of the invention or a VL domain having an amino acid sequence of a light chain expressed by a cell line expressing an antibody of the invention.

The present invention also provides antibodies that comprise, or alternatively consist of, variants (including derivatives) of the antibody molecules (e.g., the VH domains and/or VL domains) described herein, which antibodies immunospecifically bind antigen polypeptides of the invention. Standard techniques known to those of skill in the art can be used to introduce mutations in the nucleotide sequence encoding a molecule of the invention, including, for example, site-directed mutagenesis and PCR-mediated mutagenesis which result in amino acid substitutions. Preferably, the variants (including derivatives) encode less than 50 amino acid substitutions, less than 40 amino acid substitutions, less than 30 amino acid substitutions, less than 25 amino acid substitutions, less than 20 amino acid substitutions, less than 15 amino acid substitutions, less than 10 amino acid substitutions, less than 5 amino acid substitutions, less than 4 amino acid substitutions, less than 3 amino acid substitutions, or less than 2 amino acid substitutions relative to the reference VH domain, VHCDR1, VHCDR2, VHCDR3, VL domain, VLCDR1, VLCDR2, or VLCDR3. A “conservative amino acid substitution” is one in which the amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity (e.g., the ability to bind antigen polypeptides of the invention).

For example, it is possible to introduce mutations only in framework regions or only in CDR regions of an antibody molecule. Introduced mutations may be silent or neutral missense mutations, i.e., have no, or little, effect on an antibody's ability to bind antigen. These types of mutations may be useful to optimize codon usage, or improve a hybridoma's antibody production. Alternatively, non-neutral missense mutations may alter an antibody's ability to bind antigen. The location of most silent and neutral missense mutations is likely to be in the framework regions, while the location of most non-neutral missense mutations is likely to be in CDR, though this is not an absolute requirement. One of skill in the art would be able to design and test mutant molecules with desired properties such as no alteration in antigen binding activity or alteration in binding activity (e.g., improvements in antigen binding activity or change in antibody specificity). Following mutagenesis, the encoded protein may routinely be expressed and the functional and/or biological activity of the encoded protein, (e.g., ability to immunospecifically bind antigen polypeptides of the invention) can be determined using techniques described herein or by routinely modifying techniques known in the art.

In a specific embodiment, an antibody of the invention (including a molecule comprising, or alternatively consisting of, an antibody fragment or variant thereof), that immunospecifically binds antigen polypeptides of the invention, comprises, or alternatively consists of, an amino acid sequence encoded by a nucleotide sequence that hybridizes to a nucleotide sequence that is complementary to that encoding one of the VH or VL domains expressed by one or more cell lines expressing an antibody of the invention. Hybridization may occur under stringent conditions, under highly stringent conditions, under other stringent hybridization conditions which are known to those of skill in the art (see above, and, for example, Ausubel F M et al., eds. 1989. Current Protocols in Molecular Biology, Vol. I, Green Publishing Associates, Inc. and John Wiley & Sons, Inc., New York at pages 6.3.1-6.3.6 and 2.10.3). Nucleic acid molecules encoding these antibodies are also provided by the present invention.

Additionally, the term “antibody” as used herein encompasses chimeric antibodies that bind antigen polypeptides of the invention. Chimeric antibodies that bind antigen polypeptides of the invention for use in the methods of the invention have the binding characteristics of the antibodies described above. By “chimeric” antibodies is intended antibodies that are most preferably derived using recombinant deoxyribonucleic acid techniques and which comprise both human (including immunologically “related” species, e.g., chimpanzee) and non-human components, or components of two or more classes (IgG, IgM, IgE, IgA, IgD, etc.). A non-human source can be any vertebrate source that can be used to generate antibodies to antigen polypeptides of the invention. Such non-human sources include, but are not limited to, rodents (e.g., rabbit, rat, mouse, etc.; see, for example, U.S. Pat. No. 4,816,567, herein incorporated by reference) and non-human primates (e.g., Old World Monkey, Ape, etc.; see, for example, U.S. Pat. Nos. 5,750,105 and 5,756,096; herein incorporated by reference). As used herein, the phrase “immunologically active” when used in reference to chimeric antibodies means a chimeric antibody that binds antigen polypeptides of the invention.

It is well known within the art that polypeptides, or fragments or variants thereof, with similar amino acid sequences often have similar structure and many of the same biological activities. Thus, in one embodiment, an antibody (including a molecule comprising, or alternatively consisting of, an antibody fragment or variant thereof), that immunospecifically binds antigen polypeptides of the invention, comprises, or alternatively consists of, a VH domain having an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical, to the amino acid sequence of a VH domain of a heavy chain expressed by a cell line expressing an antibody of the invention.

In another embodiment, an antibody (including a molecule comprising, or alternatively consisting of, an antibody fragment or variant thereof), that immunospecifically binds antigen polypeptides of the invention, comprises, or alternatively consists of, a VL domain having an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical, to the amino acid sequence of a VL domain of a light chain expressed by a cell line expressing an antibody of the invention.

The antibodies of the invention include derivatives that are modified, i.e., by the covalent attachment of any type of molecule to the antibody. For example, but not by way of limitation, the antibody derivatives include antibodies that have been modified, e.g., by glycosylation, acetylation, phosphylation, phosphorylation, amidation, derivatization by known protecting/blocking groups, proteolytic cleavage, linkage to a cellular ligand or other protein, etc.

Furthermore, if desired, non-classical amino acids or chemical amino acid analogs can be introduced as substitutions and/or additions into the sequence of one, any, both, several or all of the polypeptides of the complex, where the cell in which the antibody is produced has the capacity to make and use such amino acid analogs. Non-classical amino acids include, but are not limited to, the D-isomers of the common amino acids, fluoro-amino acids, designer amino acids such as beta-methyl amino acids, C gamma-methyl amino acids, N gamma-methyl amino acids, and amino acid analogs in general.

Examples of non-classical amino acids include: alpha-aminocaprylic acid, Acpa; (S)-2-aminoethyl-L-cysteine.HCl, Aecys; aminophenylacetate, Afa; 6-amino hexanoic acid, Ahx; gamma-amino isobutyric acid and alpha-aminoisobytyric acid, Aiba; alloisoleucine, Aile; L-allylglycine, Mg; 2-amino butyric acid, 4-aminobutyric acid, and alpha-aminobutyric acid, Aba; p-aminophenylalanine, Aphe; b-alanine, Bal; p-bromophenylalaine, Brphe; cyclohexylalanine, Cha; citrulline, Cit; beta-chloroalanine, Clala; cycloleucine, Cle; p-cholorphenylalanine, Clphe; cysteic acid, Cya; 2,4-diaminobutyric acid, Dab; 3-amino propionic acid and 2,3-diaminopropionic acid, Dap; 3,4-dehydroproline, Dhp; 3,4-dihydroxylphenylalanine, Dhphe; p-fluorophenylalanine, Fphe; D-glucoseaminic acid, Gaa; homoarginine, Hag; delta-hydroxylysine.HCl, Hlys; DL-beta-hydroxynorvaline, Hnyl; homoglutamine, Hog; homophenylalanine, Hoph; homoserine, Hos; hydroxyproline, Hpr; p-iodophenylalanine, Iphe; isoserine, Ise; alpha-methylleucine, Mle; DL-methionine-5-methylsulfoniumchloide, Msmet; 3-(1-naphthyl)alanine, 1Nala; 3-(2-naphthyl)alanine, 2Nala; norleucine, Nle; N-methylalanine, Nmala; Norvaline, Nva; O-benzylserine, Obser; O-benzyltyrosine, Obtyr; O-ethyltyrosine, Oetyr; O-methylserine, Omser; O-methylthreonine, Omthr; O-methyltyrosine, Omtyr; Ornithine, Orn; phenylglycine; penicillamine, Pen; pyroglutamic acid, Pga; pipecolic acid, Pip; sarcosine, Sar; t-butylglycine; t-butylalanine; 3,3,3-trifluoroalanine, Tfa; 6-hydroxydopa, Thphe; L-vinylglycine, Vig; (−)-(2R)-2-amino-3-(2-aminoethylsulfonyl)propanoic acid dihydroxochloride, Aaspa; (2S)-2-amino-9-hydroxy-4,7-dioxanonanoic acid, Ahdna; (2S)-2-amino-6-hydroxy-4-oxahexanoic acid, Ahoha; (−)-(2R)-2-amino-3-(2-hydroxyethylsulfonyl)propanoic acid, Ahsopa; (−)-(2R)-2-amino-3-(2-hydroxyethylsulfanyl)propanoic acid, Ahspa; (2S)-2-amino-12-hydroxy-4,7,10-trioxadodecanoic acid, Ahtda; (2S)-2,9-diamino-4,7-dioxanonanoic acid, Dadna; (2S)-2,12-diamino-4,7,10-trioxadodecanoic acid, Datda; (S)-5,5-difluoronorleucine, Dfnl; (S)-4,4-difluoronorvaline, Dfnv; (3R)-1-1-dioxo-[1,4]thiaziane-3-carboxylic acid, Dtca; (S)-4,4,5,5,6,6,6-heptafluoronorleucine, Hfnl; (S)-5,5,6,6,6-pentafluoronorleucine, Pfnl; (S)-4,4,5,5,5-pentafluoronorvaline, Pfnv; and (3R)-1,4-thiazinane-3-carboxylic acid, Tca. Furthermore, the amino acid can be D (dextrorotary) or L (levorotary). For a review of classical and non-classical amino acids, see Sandberg et al. (Sandberg M et al. 1998. J Med. Chem. vol. 41(14): pp. 2481-91).

### Assays

Antibody Binding

The antibodies of the invention may be assayed for immunospecific binding by any method known in the art. The immunoassays which can be used include but are not limited to competitive and non-competitive assay systems using techniques such as BIAcore analysis, FACS (Fluorescence activated cell sorter) analysis, immunofluorescence, immunocytochemistry, western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few. Such assays are routine and well known in the art (see, e.g., Ausubel et al, eds, 1994, Current Protocols in Molecular Biology, Vol. 1, John Wiley & Sons, Inc., New York, which is incorporated by reference herein in its entirety). Exemplary immunoassays are described briefly below, but are not intended by way of limitation.

Immunoprecipitation protocols generally comprise lysing a population of cells in a lysis buffer such as RIPA buffer (1% NP-40 or Triton X-100, 1% sodium deoxycholate, 0.1% SDS, 0.15 M NaCl, 0.01 M sodium phosphate at pH 7.2, 1% Trasylol) supplemented with protein phosphatase and/or protease inhibitors (e.g., EDTA, PMSF, aprotinin, sodium vanadate), adding the antibody (such as, for example, but not limited to, EGX-P-E9) of interest to the cell lysate, incubating for a period of time (e.g., 1-4 hours) at 4 degrees C., adding protein A and/or protein G sepharose beads to the cell lysate, incubating for about an hour or more at 4 degrees C., washing the beads in lysis buffer and resuspending the beads in SDS/sample buffer. The ability of the antibody of interest to immunoprecipitate a particular antigen can be assessed by, e.g., western blot analysis. One of skill in the art would be knowledgeable as to the parameters that can be modified to increase the binding of the antibody to an antigen and decrease the background (e.g., pre-clearing the cell lysate with sepharose beads). For further discussion regarding immunoprecipitation protocols see, e.g., Ausubel et al, eds, 1994, Current Protocols in Molecular Biology, Vol. 1, John Wiley & Sons, Inc., New York at 10.16.1, incorporated by reference herein.

Western blot analysis generally comprises preparing protein samples, electrophoresis of the protein samples in a polyacrylamide gel (e.g., 8%-20% SDS-PAGE depending on the molecular weight of the antigen), transferring the protein sample from the polyacrylamide gel to a membrane such as nitrocellulose, PVDF or nylon, blocking the membrane in blocking solution (e.g., PBS with 3% BSA or non-fat milk), washing the membrane in washing buffer (e.g., PBS-Tween 20), blocking the membrane with primary antibody diluted in blocking buffer, washing the membrane in washing buffer, blocking the membrane with a secondary antibody (which recognizes the primary antibody, e.g., an anti-human antibody) conjugated to an enzymatic substrate (e.g., horseradish peroxidase or alkaline phosphatase) or radioactive molecule (e.g., sup.32.P or sup.125.I) diluted in blocking buffer, washing the membrane in wash buffer, and detecting the presence of the antigen. One of skill in the art would be knowledgeable as to the parameters that can be modified to increase the signal detected and to reduce the background noise. For further discussion regarding western blot protocols see, e.g., Ausubel et al, eds, 1994, Current Protocols in Molecular Biology, Vol. 1, John Wiley & Sons, Inc., New York at 10.8.1, incorporated by reference herein.

ELISAs comprise preparing antigen, coating the well of a 96 well microtiter plate with the antigen, adding the antibody of interest conjugated to a detectable compound such as an enzymatic substrate (e.g., horseradish peroxidase or alkaline phosphatase) to the well and incubating for a period of time, and detecting the presence of the antigen. In ELISAs the antibody of interest does not have to be conjugated to a detectable compound; instead, a second antibody (which recognizes the antibody of interest) conjugated to a detectable compound may be added to the well. Further, instead of coating the well with the antigen, the antibody may be coated to the well. In this case, a second antibody conjugated to a detectable compound may be added following the addition of the antigen of interest to the coated well. One of skill in the art would be knowledgeable as to the parameters that can be modified to increase the signal detected as well as other variations of ELISAs known in the art. For further discussion regarding ELISAs see, e.g., Ausubel et al, eds, 1994, Current Protocols in Molecular Biology, Vol. 1, John Wiley & Sons, Inc., New York at 11.2.1, incorporated by reference herein.

As an example, ELISAs may comprise preparing antigen, coating the well, for example, of a 96 well microtiter plate with the antigen, adding the antibody of interest conjugated to a detectable compound, such as, for example, but not limited to, an enzymatic substrate (e.g., horseradish peroxidase or alkaline phosphatase) to the well and incubating for a period of time, and detecting the presence of the antigen. In ELISAs the antibody of interest does not have to be conjugated to a detectable compound; instead, a second antibody (which recognizes the antibody of interest) conjugated to a detectable compound may be added to the well. Further, instead of coating the well with the antigen, the antibody may be coated to the well. In this case, a second antibody conjugated to a detectable compound may be added following the addition of the antigen of interest to the coated well. One of skill in the art would be knowledgeable as to the parameters that can be modified to increase the signal detected as well as other variations of ELISAs known in the art. For further discussion regarding ELISAs see, e.g., Ausubel et al, eds, 1994, Current Protocols in Molecular Biology, Vol. 1, John Wiley & Sons, Inc., New York at 11.2.1, incorporated by reference herein.

The binding affinity of an antibody to an antigen and the off-rate of an antibody-antigen interaction can be determined by any method known to one of ordinary skill in the art, such as competitive binding assays. One example of a competitive binding assay is a radioimmunoassay comprising the incubation of labeled antigen (e.g., sup.3.H or sup.125.I), or fragment or variant thereof, with the antibody of interest in the presence of increasing amounts of unlabeled antigen, and the detection of the antibody bound to the labeled antigen. The affinity of the antibody of interest for the antigen of the invention and the binding off-rates can be determined from the data by scatchard plot analysis. Competition with a second antibody can also be determined using radioimmunoassays. In this case, the antigen is incubated with antibody of interest conjugated to a labeled compound (e.g., compound labeled with sup.3.H or sup.125.I) in the presence of increasing amounts of an unlabeled second antibody. This kind of competitive assay between two antibodies, may also be used to determine if two antibodies bind the same, closely associated (e.g., overlapping) or different epitopes.

In a preferred embodiment, BIAcore kinetic analysis is used to determine the binding on and off rates of antibodies (including antibody fragments or variants thereof) to antigen of the current invention. BIAcore kinetic analysis comprises analyzing the binding and dissociation of antibodies from chips with immobilized antigen on their surface.

Binding of an antibody of the present invention to antigen of the present invention, for example, can be analyzed by BIAcore analysis. Either antigen of present invention, to which one wants to know the affinity of an antibody of the invention, or antibody of the invention, such as, for example, but not limited to, EGX-P-E9, can be covalently immobilized to a BIAcore sensor chip (CM5 chip), for example, but not limited to, via amine groups using, for example, N-ethyl-N′-(dimethylaminopropyl)carboiimide/N-hydroxysuccinimide chemistry. Various dilutions of antibodies of the invention or antigen of the invention, to which one wants to know the affinity, respectively, are flowed over the derivatized CM5 chip in flow cells, for example, at 15 microliters per minute, for example, for a total volume of 50 microliters. The amount of bound protein is determined during washing of the flow cell, for example, with HBS buffer (10 mM HEPES, pH7.4, 150 mM NaCl, 3.4 mM EDTA, 0.005% surfactant p20). Binding specificity for the protein of interest is determined by competition with soluble competitor in the presence the protein of interest.

The flow cell surface can be regenerated by displacing bound protein by washing, for example, with 20 microliters of 10 mM glycine-HCl, pH2.3. For kinetic analysis, the flow cells are tested at different flow rates and different polypeptide densities on the CM5 chip. The on-rates and off-rates can be determined using the kinetic evaluation program in a BIAevaluation 3 software.

### Neutralization Assays

Neutralization assays, i.e. the determination of whether antibodies or antisera generated by immunization of vertebrates, preferably mammals, such as, for example, but not limited to mice, rabbits, or primates, with antigen of the present invention, have viral neutralizing activity, may be performed by any method known in the art. Such methods include, as non-limiting examples, those described in detail by Dey et al. 2007 (Dey et al., 2007. Characterization of Human Immunodeficiency Virus Type 1 Monomeric and Trimeric gp120 Glycoproteins Stabilized in the CD4-Bound State: Antigenicity, Biophysics, and Immunogenicity. J Virol 81(11): 5579-5593) and Beddows et al., 2006 (Beddows et al., 2007. A comparative immunogenicity study in rabbits of disulfide-stabilized proteolytically cleaved, soluble trimeric human immunodeficiency virus type 1 gp140, trimeric cleavage-defective gp140 and momomeric gp120. Virol 360: 329-340).

### Cellular Protein Expression

Methods of recombinant cellular expression of heterologous protein are well known in the art. In eukaryotic protein expression systems, high yields of post-translationally modified and correctly folded protein can often be achieved. Purification systems are well established and commercially available.

There is also a vast literature on the expression of antibodies in mammalian cell culture that recognize the importance of post-transcriptional factors that affect the folding and assembly reactions/processes, including several chaperones or foldases (see, for example, Dinnis & James, 2005. Engineering mammalian cell factories for improved recombinant monoclonal antibody production: lessons from nature? Biotechnol Bioeng 20; 91(2):180-9).

### Expression of DNA Encoding Polypeptides

Immunoglobulin light and membrane-bound heavy chains, other proteins of the B cell receptor complex, and proteins required for downstream signaling are expressed by standard methods known to one of ordinary skill in the art.

Source of DNA

Any eukaryotic cell can serve as the nucleic acid source for molecular cloning. A nucleic acid sequence encoding a protein or domain to be engineered and/or expressed may be isolated from sources including eukaryotic, multi-cellular, animal, vertebrate, mammalian, human, porcine, bovine, feline, equine, canine, avian, etc.

The DNA may be obtained by standard procedures known in the art from cloned DNA (e.g., a DNA “library”), by chemical synthesis, by cDNA cloning, by the cloning of genomic DNA, or fragments thereof, purified from the desired cell (see e.g., Sambrook et al., 1985. Glover (ed.). MRL Press, Ltd., Oxford, U.K.; vol. I, II). The DNA may also be obtained by reverse transcribing cellular RNA, prepared by any of the methods known in the art, such as random- or poly A-primed reverse transcription. Such DNA may be amplified using any of the methods known in the art, including PCR and 5′ RACE techniques (Weis J. H. et al., 1992. Trends Genet. 8(8): 263-4; Frohman M A, 1994. PCR Methods Appl. 4(1): S40-58).

Whatever the source, the gene should be molecularly cloned into a suitable vector for propagation of the gene. Additionally, the DNA may be cleaved at specific sites using various restriction enzymes, DNAse may be used in the presence of manganese, or the DNA can be physically sheared, as for example, by sonication. The linear DNA fragments can then be separated according to size by standard techniques, such as agarose and polyacrylamide gel electrophoresis and column chromatography.

Cloning

Any method known to one of ordinary skill in the art may also be used to obtain coding sequences and sequences that regulate the rate of transcription of the DNA clone, such as, for example, genomic cloning approaches (see, for example, Fujimaki et al., 1998. The gene for human protein Z is localized to chromosome 13 at band q34 and is coded by eight regular exons and one alternative exon. Biochemistry 37(19):6838-46; Ikeno et al., 1998. Construction of YAC-based mammalian artificial chromosomes. Nat Biotechnol 16(5):431-9; Ni et al., 2009. Selective gene amplification for high-throughput sequencing. Recent Pat DNA Gene Seq 3(1):29-38; Altshuler et al., 2008. Genetic mapping in human disease. Science 322(5903):881-8); Hakomori 1999. Antigen structure and genetic basis of histo-blood groups A, B and O: their changes associated with human cancer. Biochim Biophys Acta 1473(1):247-66).

Furthermore, identification of specific DNA fragment(s) containing the desired gene may be accomplished in a number of ways. For example, clones can be isolated by using PCR techniques that may either use two oligonucleotides specific for the desired sequence, or a single oligonucleotide specific for the desired sequence, using, for example, the 5′ RACE system (Cale J M et al., 1998. Methods Mol. Biol. 105: 351-71; Frohman M A, 1994. PCR Methods Appl. 4(1): S40-58). The oligonucleotides may or may not contain degenerate nucleotide residues. Alternatively, if a portion of a gene or its specific RNA or a fragment thereof is available and can be purified and labeled, the generated DNA fragments may be screened by nucleic acid hybridization to the labeled probe (e.g. Benton and Davis, 1977. Science 196(4286): 180-2). Those DNA fragments with substantial homology to the probe will hybridize. It is also possible to identify the appropriate fragment by restriction enzyme digestion(s) and comparison of fragment sizes with those expected according to a known restriction map if such is available. Further selection can be carried out on the basis of the properties of the gene.

The presence of the desired gene may also be detected by assays based on the physical, chemical, or immunological properties of its expressed product. For example, cDNA clones, or DNA clones which hybrid-select the proper mRNAs, can be selected and expressed to produce a protein that has, for example, similar or identical electrophoretic migration; isoelectric focusing behavior, proteolytic digestion maps, hormonal or other biological activity, binding activity, or antigenic properties as known for a protein.

Using an antibody to a known protein, other proteins may be identified by binding of the labeled antibody to expressed putative proteins, for example, in an ELISA (enzyme-linked immunosorbent assay)-type procedure. Further, using a binding protein specific to a known protein, other proteins may be identified by binding to such a protein either in vitro or a suitable cell system, such as the yeast-two-hybrid system (see e.g. Clemmons D R, 1993. Mol. Reprod. Dev. 35: 368-74; Loddick S A, 1998 et al. Proc. Natl. Acad. Sci., U.S.A. 95:1894-98).

A gene can also be identified by mRNA selection using nucleic acid hybridization followed by in vitro translation. In this procedure, fragments are used to isolate complementary mRNAs by hybridization. Such DNA fragments may represent available, purified DNA of another species (e.g., Drosophila, mouse, human). Immunoprecipitation analysis or functional assays (e.g. aggregation ability in vitro, binding to receptor, etc.) of the in vitro translation products of the isolated products of the isolated mRNAs identifies the mRNA and, therefore, the complementary DNA fragments that contain the desired sequences.

In addition, specific mRNAs may be selected by adsorption of polysomes isolated from cells to immobilized antibodies specifically directed against protein. A radiolabeled cDNA can be synthesized using the selected mRNA (from the adsorbed polysomes) as a template. The radiolabeled mRNA or cDNA may then be used as a probe to identify the DNA fragments from among other genomic DNA fragments.

Alternatives to isolating the genomic DNA include, chemically synthesizing the gene sequence itself from a known sequence or making cDNA to the mRNA, which encodes the protein. For example, RNA for cDNA cloning of the gene can be isolated from cells that express the gene.

Cloning Vectors

The identified and isolated gene can then be inserted into an appropriate cloning or expression vector. A large number of vector-host systems known in the art may be used. Possible vectors include plasmids or modified viruses, but the vector system must be compatible with the host cell used. Such vectors include bacteriophages such as lambda derivatives, or plasmids such as PBR322 or pUC plasmid derivatives or the Bluescript vector (Stratagene).

The insertion into a cloning vector can, for example, be accomplished by ligating the DNA fragment into a cloning vector that has complementary cohesive termini. However, if the complementary restriction sites used to fragment the DNA are not present in the cloning vector, the ends of the DNA molecules may be enzymatically modified. Alternatively, any site desired may be produced by ligating nucleotide sequences (linkers) onto the DNA termini; these ligated linkers may comprise specific chemically synthesized oligonucleotides encoding restriction endonuclease recognition sequences. Furthermore, the gene and/or the vector may be amplified using PCR techniques and oligonucleotides specific for the termini of the gene and/or the vector that contain additional nucleotides that provide the desired complementary cohesive termini. In alternative methods, the cleaved vector and a gene may be modified by homopolymeric tailing (Cale J M et al., 1998. Methods Mol. Biol. 105: 351-71). Recombinant molecules can be introduced into host cells via transformation, transfection, infection, electroporation, etc., so that many copies of the gene sequence are generated.

Basic strategies and emerging techniques for the identification and establishment of independent transgenic mouse lines, including the use of specific DNA constructs, are discussed in Haruyama et al., 2009 (Haruyama et al., 2009. Overview: engineering transgenic constructs and mice. Curr Protoc Cell Biol Chapter 19:Unit 19.10).

Preparation of DNA

In specific embodiments, transformation of host cells with recombinant DNA molecules that incorporate an isolated gene, cDNA, or synthesized DNA sequence enables generation of multiple copies of the gene. Thus, the gene may be obtained in large quantities by growing transformants, isolating the recombinant DNA molecules from the transformants and, when necessary, retrieving the inserted gene from the isolated recombinant DNA.

The sequences provided by the instant invention include those nucleotide sequences encoding substantially the same amino acid sequences as found in native proteins, and those encoded amino acid sequences with functionally equivalent amino acids, as well as those encoding other derivatives or analogs, as described below for derivatives and analogs.

Engineering of the Immunoglobulin Heavy Chain

The portion of the cloned immunoglobulin heavy chain, so far as it is not cloned in the membrane-bound form as described above, is engineered to bind the cytoplasmic membrane and functionally interact with other proteins of a B cell receptor signaling complex according to standard methods known to one of ordinary skill in the art (see, for example, Müller et al., 1989. Membrane-bound IgM obstructs B cell development in transgenic mice. Eur J Immunol 19(5):923-928)

DNA Expression Vector Constructs

The nucleotide sequence coding for the polypeptide, or for one, any, both, several or all of the polypeptides of a complex, or analogs or fragments or other derivatives thereof, can be inserted into an appropriate expansion or expression vectors, i.e., a vector which contains the necessary elements for the transcription alone, or transcription and translation, of the inserted protein-coding sequence(s). The native genes and/or their flanking sequences can also supply the necessary transcriptional and/or translational signals.

Expression of a nucleic acid sequence encoding a polypeptide or peptide fragment may be regulated by a second nucleic acid sequence so that the polypeptide is expressed in a host transformed with the recombinant DNA molecule. For example, expression of a polypeptide may be controlled by any promoter/enhancer element known in the art.

Promoters which may be used to control gene expression include, as non-limiting examples, the SV40 early promoter region, the promoter contained in the 3′ long terminal repeat of Rous sarcoma, the herpes thymidine kinase promoter, the regulatory sequences of the metallothionein gene; prokaryotic expression vectors such as the β-lactamase promoter, or the lac promoter; plant expression vectors comprising the nopaline synthetase promoter or the cauliflower mosaic virus 35S RNA promoter, and the promoter of the photosynthetic enzyme ribulose biphosphate carboxylase; promoter elements from yeast or other fungi such as the Gal 4 promoter, the alcohol dehydrogenase promoter, phosphoglycerol kinase promoter, alkaline phosphatase promoter, and the following animal transcriptional control regions, which exhibit tissue specificity and have been utilized in transgenic animals: elastase I gene control region which is active in pancreatic acinar cells (Swift et al., 1984. Cell 38: 639-46); a gene control region which is active in pancreatic beta cells (Hanahan D, 1985. Nature 315: 115-22), an immunoglobulin gene control region which is active in lymphoid cells (Grosschedl R et al., 1984. Cell; 38: 647-58), mouse mammary tumor virus control region which is active in testicular, breast, lymphoid and mast cells (Leder A et al., 1986. Cell; 45: 485-95), albumin gene control region which is active in liver (Pinkert C A et al., 1987. Genes Dev. 1: 268-76), alpha-fetoprotein gene control region which is active in liver (Knumlauf R et al., 1985. Mol. Cell. Biol. 5: 1639-48); alpha 1-antitrypsin gene control region which is active in the liver (Kelsey G D et al., 1987. Genes Dev. 1: 161-71), beta-globin gene control region which is active in myeloid cells (Magram J et al., 1985 Nature 315: 338-40); myelin basic protein gene control region which is active in oligodendrocyte cells in the brain (Readhead C et al., 1987 Cell 48: 703-12); myosin light chain-2 gene control region which is active in skeletal muscle (Shani M, 1985. Nature 314: 283-86), and gonadotropic releasing hormone gene control region which is active in the hypothalamus (Mason A J et al., 1986. Science 234: 1372-78).

In a specific embodiment, a vector is used that comprises a promoter operably linked to a gene nucleic acid, one or more origins of replication, and, optionally, one or more selectable markers (e.g., an antibiotic resistance gene).

Vectors containing gene inserts can be identified by three general approaches: (a) identification of specific one or several attributes of the DNA itself, such as, for example, fragment lengths yielded by restriction endonuclease treatment, direct sequencing, PCR, or nucleic acid hybridization; (b) presence or absence of “marker” gene functions; and, where the vector is an expression vector, (c) expression of inserted sequences. In the first approach, the presence of a gene inserted in a vector can be detected, for example, by sequencing, PCR or nucleic acid hybridization using probes comprising sequences that are homologous to an inserted gene. In the second approach, the recombinant vector/host system can be identified and selected based upon the presence or absence of certain “marker” gene functions (e.g., thymidine kinase activity, resistance to antibiotics, transformation phenotype, occlusion body formation in baculovirus, etc.) caused by the insertion of a gene in the vector. For example, if the gene is inserted within the marker gene sequence of the vector, recombinants containing the insert an identified by the absence of the marker gene function. In the third approach, recombinant expression vectors can be identified by assaying the product expressed by the recombinant expression vectors containing the inserted sequences. Such assays can be based, for example, on the physical or functional properties of the protein in in vitro assay systems, for example, binding with anti-protein antibody.

Once a particular recombinant DNA molecule is identified and isolated, several methods known in the art may be used to propagate it. Once a suitable host system and growth conditions are established, recombinant expression vectors can be propagated and prepared in quantity. Some of the expression vectors that can be used include human or animal viruses such as vaccinia virus or adenovirus; insect viruses such as baculovirus; and plasmid and cosmid DNA vectors.

Once a recombinant vector that directs the expression of a desired sequence is identified, the gene product can be analyzed. This is achieved by assays based on the physical or functional properties of the product, including radioactive labeling of the product followed by analysis by gel electrophoresis, immunoassay, etc.

As described, for example, in detail in Meffre & Nussenzweig, 2002, Yu et al., 1999, and Misulovin et al., 2001 (Meffre & Nussenzweig, 2002. Deletion of immunoglobulin beta in developing B cells leads to cell death. Proc Natl Acad Sci USA 99(17):11334-11339; Yu et al., 1999. Continued RAG expression in late stages of B cell development and no apparent re-induction after immunization. Nature 400: 682-687; and Misulovin et al., 2001. A rapid method for targeted modification and screening of recombinant bacterial artificial chromosome. J. Immunol. Methods 257: 99-105), bacterial artificial chromosomes (BAC) may also be used to express proteins, for example, but not limited to, under the control of tissue and/or cell-type specific promoters/regulatory sequences.

### Methods of Knocking Out, or Knocking Down Endogenous Immunoglobulin Expression

Many methods are known in the art of knocking out or modulating the expression of a known gene or genomic DNA sequence. Examples of such methods include, but are not limited to, siRNA targeting, targeted gene knock-out, transfection with a transcriptional factor, and site-specific cleavage of the DNA strands encoding endogenous immunoglobulin protein. In principle, any molecular biology, cell biology, or selection method can be used to reduce the expression level of the endogenous immunoglobulin protein.

Gene Targeting Used to Knock Out Endogenous Immunoglobulin Expression

Disruption of the genome can be obtained by gene targeting or the knock-out technique. The generation of knock-out cells is a well-described technique for eradicating expression of endogenous proteins, and knock-out in a cell-line (CHO cells) was recently described (Yamane-Ohnuki et al, 2008. Methods for producing modified glycoproteins. U.S. Pat. No. 7,326,681; Yamane-Ohnuki et al., 2004. Establishment of FUT8 knockout Chinese hamster ovary cells: an ideal host cell line for producing completely defucosylated antibodies with enhanced antibody-dependent cellular cytotoxicity. Biotechnol. Bioeng. 87 (5):614-622). A genomic knockout plasmid was generated and transfected into CHO cells. By homologous recombination the targeted gene in the CHO cells was disrupted.

Knockout is accomplished, beginning with the construction of a DNA construct, such as, for example, but not limited to, a plasmid or a bacterial artificial chromosome, and proceeding to cell culture. Individual cells are genetically transformed with the DNA construct. The DNA construct is engineered to recombine with the target gene, which is accomplished by incorporating sequences from the gene itself into the construct. Recombination then occurs in the region of that sequence within the gene, resulting in the insertion of a foreign sequence to disrupt the gene. With its sequence interrupted, the altered gene in most cases will be translated into a nonfunctional protein, if it is translated at all. Where the goal is to create a transgenic animal that has the altered gene, embryonic stem cells are genetically transformed and inserted into early embryos. Resulting animals with the genetic change in their germline cells can then often pass the gene knockout to future generations. These and other related and relevant methods are described in U.S. Pat. Nos. 7,491,868; 6,562,624; and 6,414,219; United States Patent Application Numbers 20070056052, 20070250939, 20060015954, 20050026292, 20040261139, 20040158884, 20040132145; and in Johzuka-Hisatomi, Terada, & Iida, 2008 (“Efficient transfer of base changes from a vector to the rice genome by homologous recombination: involvement of heteroduplex formation and mismatch correction.” Nucleic Acids Res 36(14):4727-35); Iida & Terada, 2004 (“A tale of two integrations, transgene and T-DNA: gene targeting by homologous recombination in rice.” Curr Opin Biotechnol 15(2):132-8); Belancio, Hedges, & Deininger, 2008 (“Mammalian non-LTR retrotransposons: for better or worse, in sickness and in health.” Genome Res 18(3):343-58); Ostertag, Madison, & Kano, 2007 (“Mutagenesis in rodents using the L1 retrotransposon.” Genome Biol 8 Suppl 1:S16); Tronche et al., 2002 (“When reverse genetics meets physiology: the use of site-specific recombinases in mice.” FEBS Lett 529(1):116-21); and Cohen-Tannoudji & Babinet, 1998 (“Beyond ‘knock-out’mice: new perspectives for the programmed modification of the mammalian genome.” Mol Hum Reprod. 4(10):929-38); all of foregoing are incorporated in their entirety by references herein.

A conditional knockout allows gene deletion in a tissue or time specific manner. This is done by introducing short sequences called loxP sites around the gene. These sequences are introduced into the germ-line via the same mechanism as a knock-in (knock-in is similar to knock-out, but instead it replaces a gene with another instead of deleting it). This germ-line can then be crossed with another germline containing Cre-recombinase, a bacterial enzyme that recognizes these sequences and recombines them, deleting the gene flanked by these sites. These and other related and relevant methods are described in U.S. Pat. Nos. 7,145,056, 7,112,715, 6,734,295, 6,596,508, and 5,434,066, United States patent application numbers 20050289659 and 20020106720, and in Turakainen et al., 2009 (“Transposition-based method for the rapid generation of gene-targeting vectors to produce Cre/Flp-modifiable conditional knock-out mice.” PLoS ONE 4(2):e4341); García-Otín & Guillou, 2006 (“Mammalian genome targeting using site-specific recombinases.” Front Biosci 11:1108-36); Sykes & Kamps, 2003 (“Estrogen-regulated conditional oncoproteins: tools to address open questions in normal myeloid cell function, normal myeloid differentiation, and the genetic basis of differentiation arrest in myeloid leukemia.” Leuk Lymphoma 44(7):1131-9); and Hohenstein et al., 2008 (“High-efficiency Rosa26 knock-in vector construction for Cre-regulated overexpression and RNAi.” Pathogenetics 1(1):3); all of the foregoing are incorporated in their entirety by reference herein.

In diploid organisms, which contain two alleles for most genes, and may as well contain several related genes that collaborate in the same role, it may be necessary to perform additional rounds of transformation and selection, depending on the targeted protein, until every targeted gene is knocked out. Selective breeding may be required to produce homozygous knockout animals.

Random Mutagenesis Used to Knock Down or Know Out Endogenous Immunoglobulin Expression

The use of random mutagenesis to introduce genomic changes in the host cells, some of which may prevent the generation of mRNA in the host cell may also be exploited. This may be achieved by treating a population of cells with a mutagen, such as, for example, but not limited to, Ethyl Methane Sulfonate, EMS, which induces point mutations in the cells. The surviving cells may exhibit altered phenotypes, because of these mutations. The cells may be seeded in a screening format (e.g. 96-well plates) to allow isolation of clonal cell populations. Following a growth period, cells may be harvested from the wells and assayed for surface immunoglobulin expression by any standard methods known to one of ordinary skill in the art.

Knocking Down Endogenous Immunoglobulin Expression Using Targeted siRNA

Small interfering RNA (siRNA), as opposed to small activating RNA (saRNA), a class of 20-25 nucleotide-long double-stranded RNA molecules that play a variety of roles in biology, is involved in the RNA interference (RNAi) pathway, where it interferes with the expression of one or more specific genes. siRNAs have well-defined structures, comprising a short (usually 21-nt) double strand of RNA (dsRNA) with 2-nt 3′ overhangs on either end. Each strand has a 5′ phosphate group and a 3′ hydroxyl (—OH) group. The structure results from processing by an enzyme (“dicer”) that converts either long dsRNAs or small hairpin RNAs into siRNAs (Bernstein et al., 2001. Role for a bidentate ribonuclease in the initiation step of RNA interference. Nature 409: 363-6; the foregoing reference is incorporated herein in its entirety). SiRNAs can also be exogenously introduced into cells by various methods to bring about the specific knockdown of a gene of interest. Essentially any gene for which the sequence is known can thus be targeted based on sequence complementarity with an appropriately tailored siRNA. This has made siRNAs a very important tool in biomedical research and engineering.

Exogenous siRNA can be transfected into cells, which can, however, be problematic because the gene knockdown effect is only transient, particularly in rapidly dividing cells. To overcome this challenge, it is possible to allow the siRNA to be expressed by an appropriate vector, e.g., a plasmid. Stable expression of small interfering RNA, siRNA, is a new technology that enables reduction of targeted mRNA and thus suppression of targeted gene expression in mammalian cells (Brummelkamp, et al., 2002. A system for stable expression of short interfering RNAs in mammalian cells. Science 296(5567): 550-553; Mivaaishi & Taira, 2002. U6 promoter-driven siRNAs with four uridine 3′ overhangs efficiently suppress targeted gene expression in mammalian cells. Nat. Biotechnol 20(5):497-200) In essence, a loop between the two strands is introduced to produce a single transcript, which is processed into a functional siRNA. Transcription of such constructs is typically driven by an RNA polymerase III promoter, which is normally associated with transcription of small nuclear RNAs. It is assumed that the resulting siRNA transcript is then processed by the dicer enzyme. A number of individual siRNA have been generated in a strategy similar to the ones described in the references.

On occasion nonspecific effects are triggered by the experimental introduction of siRNAs. Mammalian cells may mistake an siRNA for a viral product or by-product and mount an immune response. One method to address this issue is to convert the siRNA into a microRNA, whereby it is often possible to achieve similar gene knockdown at comparatively low concentrations of resulting siRNAs. Furthermore, introduction or expression of an siRNA may cause unintended off-targeting. This, however, can be addressed by designing appropriate control experiments, and siRNA design algorithms are currently being developed to produce siRNAs free from off-targeting. Genomic expression analysis can be usedapplied to verify specificity and further refine the algorithm(s) (Birmingham et al., 2006. 3′ UTR seed matches, but not overall identity, are associated with RNAi off-targets. Nat Methods 3: 199-204; the foregoing reference is incorporated herein in its entirety). These and other related and relevant methods are described in U.S. Pat. Nos. 7,507,809; 7,393,683; and 7,361,752, in United States patent application numbers 20090060889, 20090054366, 20090054365, 20090042823, 20090036397, 20090036396, 20090035861, 20090035225, 20090023671, 20090012030, 20080300145, 20080293662, 20080274996, 20080248468, 20080241198, 20080233584, 20080220027, 20080214488, 20080214486, 20080213891, 20080213871, 20080194513, 20080171719, 20080153771, 20080120733, 20080113931, 20080081791, 20080051361, 20080022423, 20080015161, 20070298481, 20070281901, 20070275922, 20070275919, 20070249009, 20070248960, 20070218079, 20070213293, 20070203082, 20070197460, 20070179160, 20070166716, 20070155690, 20070155686, 20070149473, 20070082864, 20070081982, 20070044161, 20070042984, 20070033663, 20070025969, 20070004040, 20060287264, 20060270621, 20060263764, 20060258608, 20060223773, 20060217324, 20060189561, 20060178329, 20060172965, 20060172963, 20060172961, 20060166921, 20060166919, 20060166918, 20060115455, 20060115454, 20060105976, 20060105377, 20060089324, 20060089323, 20060088837, 20060063161, 20060058255, 20050283845, 20050266552, 20050245472, 20050229266, 20050227940, 20050208518, 20050197313, 20050186589, 20050186586, 20050181382, 20050171041, 20050166272, 20050112763, 20050074887, 20050059028, 20050059019, 20050042641, 20050026286, 20050019927, 20050003541, 20040248839, 20040248164, 20040152172, 20040096882, 20040086911, 20030153519, 20030139363, and 20020173478; and in Siomi & Siomi, 2009 (“On the road to reading the RNA-interference code.” Nature 457(7228):396-404); Ohrt & Schwille, 2008 (“siRNA modifications and sub-cellular localization: a question of intracellular transport?” Curr Pharm Des 14(34):3674-85); Pushparaj et al., 2008 (“siRNA, miRNA, and shRNA: in vivo applications. J Dent Res 87(11):992-1003; Kim & Rossi, 2008. RNAi mechanisms and applications.” Biotechniques 44(5):613-6); Merkenschlager & Wilson, 2008 (“RNAi and chromatin in T cell development and function.” Curr Opin Immunol 20(2):131-8); Stormo, 2006 (An overview of RNA structure prediction and applications to RNA gene prediction and RNAi design.” Curr Protoc Bioinformatics; Chapter 12:Unit 12.1); Rossi, 2008 (“Expression strategies for short hairpin RNA interference triggers.” Hum Gene Ther 19(4):313-7); Beaucage, 2008 (“Solid-phase synthesis of siRNA oligonucleotides.” Curr Opin Drug Discov Devel 11(2):203-16); Lavrov & Kibanov, 2007 (“Noncoding RNAs and chromatin structure.” Biochemistry (Mosc) 72(13):1422-38); Jaskiewicz & Filipowicz, 2008 (“Role of Dicer in posttranscriptional RNA silencing.” Curr Top Microbiol Immunol 320:77-97); Paddison, 2008 (“RNA interference in mammalian cell systems.” Curr Top Microbiol Immunol 320:1-19); Hawkins & Morris, 2008 (“RNA and transcriptional modulation of gene expression.” Cell Cycle 7(5):602-7); Liu et al., 2008 (“MicroRNAs: biogenesis and molecular functions.” Brain Pathol 18(1):113-21); Guan & Kiss-Toth, 2008 (“Advanced technologies for studies on protein interactomes.” Adv Biochem Eng Biotechnol 110:1-24); Ku & McManus, 2008 (“Behind the scenes of a small RNA gene-silencing pathway.” Hum Gene Ther 19(1):17-26); Lin et al., 2008 (“Intron-mediated RNA interference and microRNA (miRNA).” Front Biosci 13:2216-30); Kohonen et al., 2007 (“Avian model for B-cell immunology—new genomes and phylotranscriptomics.” Scand J Immunol 66(2-3):113-21); and Svoboda, 2007 (“Off-targeting and other non-specific effects of RNAi experiments in mammalian cells.” Curr Opin Mol Ther 9(3):248-57); all of the foregoing are incorporated in their entirety by reference herein.

Transcription Factor Engineering Used to Down-Regulate Endogenous Immunoglobulin Expression

Expression of endogenous immunoglobulin may be reduced or abolished by transcriptional down regulation of immunoglobulin mRNA. Transcription factors are designed to bind specific DNA elements in the promoter region of endogenous immunoglobulin. Zinc finger proteins are particularly well suited for such a manipulation and common procedures are reviewed in several publications (e.g. Wolfe et al., 1999. Arum. Rev. Biophys. Struct. 3:183-212; Jamieson et al., 2003. Nature Reviews, vol 2:361-368). Typically a single zinc finger binds three bases adjacent to each other on the same DNA strand and a forth base on the complementary strand. Thus, several zinc fingers can be combined in order to bind a desired DNA element. Recognition of a DNA element of 15-18 base pairs, which actually can be universal in the genome, needs a combination of 5-6 zinc fingers.

A DNA element of a specific sequence of the endogenous immunoglobulin promoter is chosen and Zinc finger proteins binding the DNA element is predicted based on available publications (Liu et al., 2001. Regulation of an endogenous locus using a panel of designed zinc finger proteins targeted to accessible chromatin regions. Activation of vascular endothelial growth factor A. J Biol Chem 276 (14): 11323-11334; Zhang et al., 2000. Synthetic zinc finger transcription factor action at an endogenous chromosomal site. Activation of the human erythropoietin gene. J Biol Chem 275 (43): 33850-33860). A synthetic gene directing the expression of a five zinc finger protein is made by PCR from overlapping oligonucleotides (Zhang et al., 2000. Synthetic zinc finger transcription factor action at an endogenous chromosomal site. Activation of the human erythropoietin gene. J Biol Chem 275 (43): 33850-33860). The plasmid encoding the synthetic gene is transfected into cells; upon binding of the engineered zinc finger protein to the endogenous immunoglobulin promoter DNA element, transcription of endogenous immunoglobulin is down-regulated. Similar methods are described in Ekker, 2008 (“Zinc finger-based knockout punches for zebrafish genes.” Zebrafish 5(2):121-3), incorporated in its entirety by reference herein.

### Expressing DNA Encoding Polypeptides in Cells of the Invention

Recombinant molecules can be introduced into host cells via transformation, transfection, infection, electroporation, etc., either so that many copies of the gene sequences are generated, or so that the proteins are transcribed and translated, and post-translationally modified where helpful or necessary, or both. Proteins can be expressed transiently or stable cell lines can be generated, each according to standard methods known to one of ordinary skill in the art.

As a non-limiting example, lentiviral vectors with the measles virus H and F glycoproteins on their surface that transduce quiescent B-cells may be used to express DNA constructs of the instant invention, including, for example, but not limited to, immunoglobulin proteins, markers, siRNAs, and saRNAs (see, as a non-limiting example, Frecha et al., 2009. Blood 114(15):3173-80).

For example, the lentiviral vector for expression of anti-HIV Env antibodies in chicken DT40 cells may be co-transfected with canine distemper virus H and F glycoprotein genes, analogously to the methods described by Frecha et al (see above). CDV H & F glycoprotein genes/DNA may be modified to match the avianized strain of the virus—the Onderstepoort strain, that infects chicken cells (Tatsu et al., 2001. J Virol 75(13): 5842-5850; Haig D A. Onderstepoort J Vet Res. 1956; 17:19-53; Frecha et al., 2009. Blood 114(15):3173-80; v. Messling et al., 2003. J Virol 77(23): 12579-12591). Alternatively, this set of genes can be expressed in expression vectors, introduced by electroporation, and transduced cells can be isolated by resistance marker selection.

Furthermore, transgenic animals that express membrane-bound forms of either IgD or IgM antibodies, or both, with the same variable domains as the neutralizing antibodies that are introduced and assayed by the methods of this invention, either constitutively, or in an inducible or tissue/cells specific manner can be generated by standard methods known to one of ordinary skill in the art (see, for example, Meffre & Nussenzweig, 2002. Deletion of immunoglobulin beta in developing B cells leads to cell death. Proc Natl Acad Sci USA 99(17):11334-11339; Yu et al., 1999. Continued RAG expression in late stages of B cell development and no apparent re-induction after immunization. Nature 400: 682-687; and Misulovin et al., 2001. A rapid method for targeted modification and screening of recombinant bacterial artificial chromosome. J. Immunol. Methods 257: 99-105).

### Assaying BCR Activation

### Analysis of Cytoplasmic Signaling Molecules

Any method known to one of ordinary skill in the art may be used to assay biochemical, biophysical, any other alterations of downstream signaling, or changes in their subcellular localization before and after exposure of one or more cells expressing one or more antibody of the invention to an antigen of the invention. Alterations induced by B cell activation that may be assayed include, for example, but not limited to, elevated or diminished enzymatic activity (e.g. tyrosine or serine/threonine kinase and phosphatase activities), protein/substrate phosphorylation or dephosphorylation, or any other kind of post-translational modifications, and association with other molecules. As non-limiting examples, G-proteins may more prevalently be associated with GTP or GDP following B cell stimulation, adapter proteins may associate with, or dissociate from, cytoskeletal/structural or enzymatic proteins. Other methods for assaying signaling downstream of B cell receptor activation include second messenger analysis, such as, for example, but not limited to, measuring intracellular calcium flux.

In a preferred embodiment, the assay requires a limited number of steps, is robust, straight-forward, and not consuming, and is therefore compatible with high-through-put analysis. Examples include standard kinase assays know to one or ordinary skill in the art and, furthermore, as a non-limiting example, the methodology described in Mahajan et al., 2006 (Mahajan et al., 2006. Cell-based kinase assay. US Patent Application 20060141549). Another non-limiting example is described by Guo et al., 2009 (Guo et al., 2009. Reagents for the Detection of Protein Acetylation Signaling Pathways. US Patent Application 20090124023).

### Ca⁺⁺ Influx Assays

A non-limiting example is use of a second messenger assay that meets the requirements for automated, high-through-put analysis/screening, such as, for example, but not limited to, the Fluo-4 NW (No Wash) Calcium Assay sold commercially available from Invitrogen. The Fluo 4NW Calcium Assay meets the requirements of automated screening (HTS) applications, does not require a quencher dye, an provides the convenience of a no-wash format. Other non-limiting examples of methods for assaying signaling molecules are described in Palmer, 2009. (Palmer, 2009. Cellular Signaling Pathway Based Assays, Reagents and Kits. US Patent Application 20090111710), which may be adapted by methods known to one of ordinary skill in the art to assaying for signaling molecules downstream of B cell receptor activation.

Molecular Devices commercializes the FlexStation-compatible Calcium Assays 3, 4, and 5 that are HTP-compatible and sensitive, fluorescence- and quench technology-based FlexStation assays for detecting changes in intracellular calcium concentration in a straightforward and homogeneous format that yields maximum signal intensity, and thereby allows accurate and non-labor intensive detection of intercellular calcium fluxes. Optimized chemistry combined with a no-wash protocol has the following benefits: (i) minimal cellular disruption (ii) reduced frequency of spontaneous calcium fluxes and unresponsive cells (iii) good results from low-expression receptors that are otherwise difficult to assay (iv) consistent and strong signals with high well-to-well uniformity and superior data quality and higher Z′-factors. After incubation with the reagents, cells are stable for several hours. Rapid analysis of the cells can be followed with detection on a FlexStation microplate reader.

This assay reduces required preparation time and increases throughput by eliminating wash steps, which eliminates potential dispensing and washing errors, or associated equipment failures, and further ensures the integrity of screening operations. This also reduces the causes for data variability, and reduces false positive and negative noise. The assay can be carried out at room temperature, which facilitates automation using stackers or robots. Larger volume packaging minimizes reagent bottle and liquid handling. The Molecular Devices assays are increasingly being used to assay functional Ca⁺⁺ influx responses in cell lines, for example in response to G protein-coupled receptor activation (Roncarati et al., 2008. Assay Drug Dev Technol 6(2):181-93; Xin et al., 2007. J Biomol Screen 12(5):705-14; Xie et al., 2007. Assay Drug Dev Technol 5(2):191-203; Lubin et al., 2006. Assay Drug Dev Technol 4(6):689-94).

### Caspase-3 Activation Assay

Non-limiting example of assays that measures/analyzes elevated or diminished enzymatic activity are Caspase-3 activity assays. High affinity binding of antigen to BCR receptors, and high signal strength, provoke apoptosis in mature B cells through downstream signals that lead to caspase-3 activation. Caspase-3 has long been identified as a key mediator of apoptosis of mammalian cells. (e.g. Tsirigotis P, Economopoulos T 2008. J Steroid Biochem Mol Biol. 108(3-5):267-71).

A non-limiting example of a Caspase-3 assay is the OncoImmunin PhiPhiLux system. OncoImmunin's PhiPhiLux reagents are peptide-based, fluorogenic substrates for apoptosis-specific caspase 3 and caspase 3-like activity assays, comprising a peptide with the DEVDGI proteolytic cleavage sequence and fluorophores, which can be used in flow cytometry of living cells, as they are able to cross intact cell membranes. To reduce noise due to biological materials' absorbance and fluorescence in the UV wavelengths, the fluorophores have both excitation and emission in the visible wavelength region, increasing sensitivity. Peptide substrates are synthesized with the complete protease recognition sequences, and two fluorophores are coupled covalently so they form non-fluorescent dimmers; in this configuration, the peptide assumes the conformation the protease(s) recognize and cleave efficiently. Peptide cleavage abolishes the dye-dye interaction, results in an increase in fluorescence and significant absorption changes, and sensitively reports in vivo proteolyic activity of the enzyme. As described above, because effective signaling may leads to apoptosis certain cell lines, an assay measuring apoptotic signaling is such cells would not contribute to the assay's ability to distinguish between primary signal strengths leading to proliferative vs. apoptotic responses, and therefore this assay is most preferably carried out with cells in which this distinction can be made, such as, for example, but not limited to, human peripheral mature naïve B cells (Kohonen P et al. 2007. Scand J Immunol 66:113-21).

### Analysis of Transcriptional Analysis

Increases or decreases in transcriptional activity may be monitored as a method of analyzing signaling downstream of B cell receptor activation. Transcription rates of genes known or discovered to be regulated by B cell receptor activation-induced signaling can be analyzed by any method known to one of ordinary skill in the art, including, for example, but not limited Northern blot analysis and RT PCR.

In a preferred embodiment, the assay requires a limited number of steps, is robust, straight-forward, and not consuming, and is therefore compatible with high-through-put analysis. Non-limiting examples include transcription factor responsive reporter gene assays, whereby DNA constructs, such as, for example, plasmids, cosmids, BACs, etc., comprising a transcription factor responsive promoter, such as, for example, a promoter comprising an NF kappa B-responsive element, that drives/regulates transcription of a reporter gene, such as, for example, a firefly or a Renilla luciferase, and any other components required for replication, selection, integration, etc., are transfected, and luciferase activity is assayed by luminescence or fluorescence before and after exposure of one or more cells expressing one or more antibody of the invention to an antigen of the invention. Cells may comprise one or more stably integrated copies of the DNA construct, stably maintained copies of the DNA construct, or the DNA construct may be available for the assay temporarily, such as, for example, but not limited to, following transient transfection.

### NFκB-Responsive Reporter Gene Assay

A non-limiting example of transcriptional analysis is the use of reporter genes under the control of promoters comprising multiple c-Rel/NFκB-responsive elements that provide highly sensitive assays with a wide dynamic ranges. These assays are applied to assay for cRel/NFκB-mediated induction of transcription, which itself can serve as a marker for cellular responses and differentiation. In response to BCR ligation and downstream signaling that leads to a proliferative response, cRel binds to the canonical NFκB binding site, and up-regulates transcription of genes under the control of promoters that contain these binding sites. Thus an assay that reports cRel-mediated up-regulation provides a functional read-out of BCR-induced signals that lead to proliferative responses. Since, due to an arrested stage of development, effective signaling may lead to apoptosis in certain cell lines, such as, for example, but not limited to, DT 40 cells, and proliferative NFκB signaling would not expected. Therefore these assays will are most preferably carried out in cells that lead to a proliferative response, such as, for example, but not limited to, human peripheral mature naïve B cells (Kohonen P et al. 2007. Scand J Immunol 66:113-21).

### DEFINITIONS, MODIFICATIONS, AND INCLUSIONS

Throughout the specification, unless the context requires otherwise, the word ‘comprise’, and variations such as ‘comprises’ and ‘comprising’, will be understood to imply the inclusion of a stated integer or step or group of integers but not to the exclusion of any other integer or step or group of integers or steps.

Any patents or publications mentioned in this specification are indicative of the levels of those skilled in the art to which the invention pertains. All publications, including but not limited to patents and patent applications, cited in this specification are herein incorporated by reference as if each individual publication were specifically and individually indicated to be incorporated by reference herein as though fully set forth.

The description/specification fully discloses the invention, including preferred embodiments thereof. Modifications and improvements of the embodiments specifically disclosed herein are within the scope of the invention. One skilled in the art will appreciate that the present invention is well adapted to carry out the objects and obtain the ends and advantages mentioned, as well as those objects, ends and advantages inherent herein. Changes therein and other uses which are encompassed within the spirit of the invention as defined by the scope of the claims will occur to those skilled in the art. It is believed that one skilled in the art can, using the description herein, utilize the present invention to its fullest extent. Therefore the individual embodiments herein are to be construed as merely illustrative and not a limitation of the scope of the present invention in any way.

### Example 1

### Flexstation Ca⁺⁺ Influx Assay in DT40

A Ca⁺⁺ influx assay used to measure primary signal strength in both DT40 cells and human peripheral mature naïve B cells is robust and sensitive. The Molecular Devices Calcium 3, 4, and 5 Assays are currently among the most suitable for high-throughput analysis. Roach et al. demonstrated that the assay is highly sensitive in culture splenic B cells from human bcl-2 transgenic mice (Roach et al. 2004. AfCS Research Reports 2 (13 BC)); Ca⁺⁺ signaling assays have also been performed in DT40 cells (Yasuda & Yamamoto, 2001. In: Methods in Molecular Biology, vol. 271: B Cell Protocols, Eds Gu H and Rajewsky K. Humana Press Inc., Totowa, N.J.); we tested Ca⁺⁺ influx in DT40 cells with the Molecular Devices Calcium 4 Assay kits or reagents.

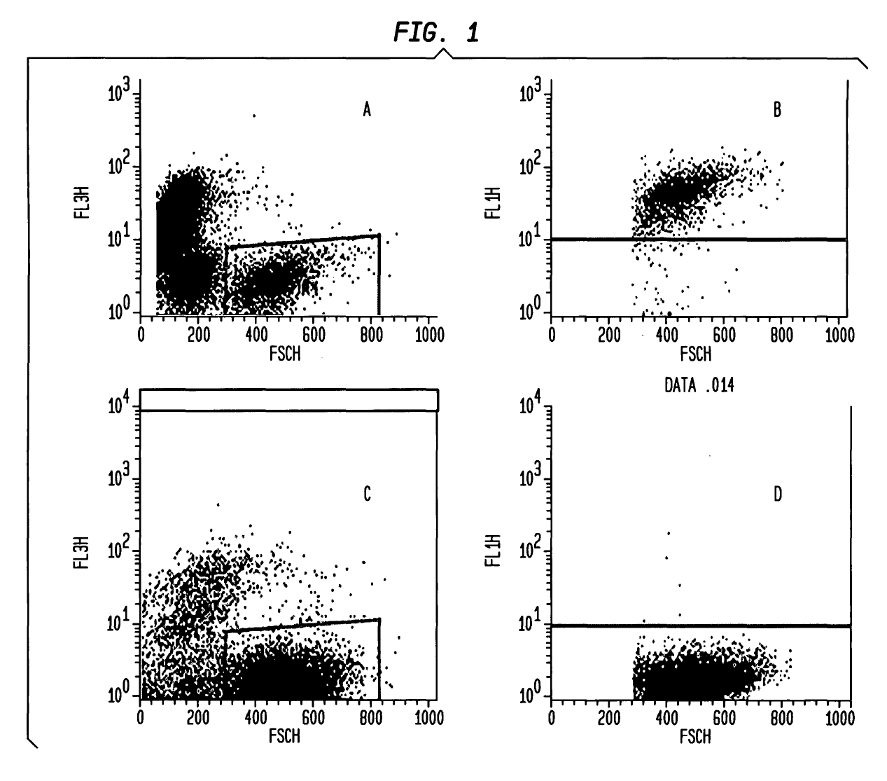

Wild-type DT40 cells expressing surface-bound IgM, and AID^{-/-}/IgH⁻/IgL⁻ cells described by Arakawa et al. (Arakawa, Hauschild, & Buerstedde, 2002. Science 295:1301-6) were cultured in RPMI (Invitrogen) with 10% v/v chicken serum, penticillin, streptomycin, and BME. Cell surface expression of IgM on DT40 cells was verified in wild-type by flow cytometry, with the AID^{-/-}/IgH⁻/IgL⁻ cells as a negative control (FIG. 1). Prior to performing the calcium influx assays, the DT40 cells were seeded in 384 well plates at a density of 50,000 cells per well in a volume of 254 Cells were loaded with an equal volume of Calcium 4 dye-buffer (Molecular Devices, Sunnyvale, Calif.) for 1 hr at 37° C. directly in 384 well plates, and kept at RT for 30 min prior to assay.

Initial experiments were performed to validate calcium flux in DT40s in response to an ionophore, ionomycin. Assays were performed using a FlexStationII. The parameters were set to an excitation wavelength of 485 nm, emission wavelength of 525 nm, emission cut-off at 515 nm, pipette height of 230 μl, a transfer volume of 10 μl, 5-fold compound concentration, and an addition speed rate of 20 μl/sec.

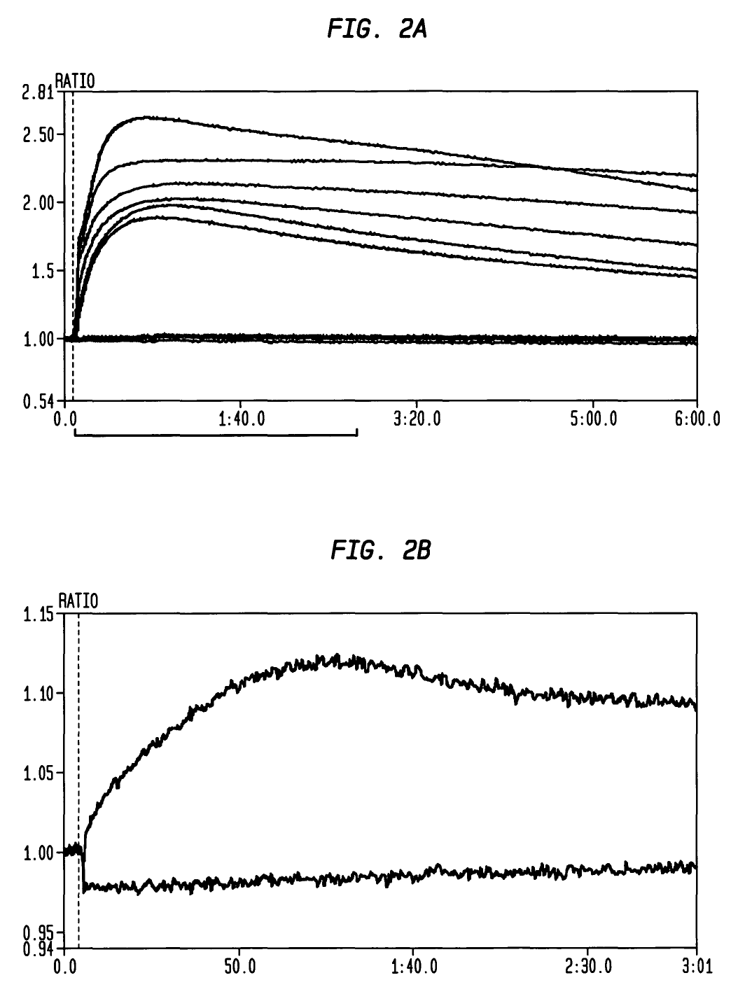

Addition of 100 μM ionomyocin dissolved in 10% DMSO in a 10 μl volume resulted in rapid influx of Ca⁺⁺ and a strong fluorescent response (FIG. 2A). The calcium response was dose dependant, demonstrating the wide dynamic range of this assay in DT40 cells. The maximum signal plateau was reached after about 40 seconds.

To validate the assay in this system, we also used a monoclonal anti-chicken IgM antibody (Southern biotechnologies, inc.) to ligate the BCR on DT40 cells in concentrations ranging from 30 ng to 5 μg per assay (60 μl total volume) and assayed for Ca⁺⁺ influx. Calcium-flux signal was recorded with a maximum of approximately 10% of the maximum ionomycin maximum that reached a plateau at approximately 90 seconds (FIG. 2B). These results demonstrate the ability of this assay to report signal strength of a BCR-ligand interaction as a quantitative fluorescent measurement useful for screening BCR specific antigens. In the course of the research program, we will substantially enhance the sensitivity of the assay to be able to detect a greater breadth of immunogen-specific signals.

### Example 2

### Expression of “Chickenized” Surface IgM in DT40 Cells

In order to screen potential HIV immunogens for their effect on B cell signaling DT40 cell lines expressing broadly neutralizing anti-HIV antibodies as surface-bound IgM are generate. We modified the human broadly neutralizing anti-HIV antibody, B 12 (SEQ ID NO 1, SEQ ID NO 3, SEQ ID NO 3, and SEQ ID NO 4) by replacing the IgG1 heavy chain C terminus with the C-terminus of chicken IgM, including the membrane anchor domain. The result is a gene that directs expression of a chimeric membrane-bound heavy chain that, co-expressed with the light chain, has b12 specificity, and that functions as part of the chicken DT40 BCR complex. This allows evaluation of B cell responses following HIV antigen binding. To ensure that observed responses result from activation of BCR complexes comprising surface-expressed anti-HIV Ab H & L chains, AID^{-/-}/IgH⁻/IgL⁻ DT40 cells were used for expression and Ca⁺⁺ assays.

WT DT40 cells were grown in RPMI supplemented with 10% heat-inactivated chicken serum, penicillin, streptomycin, and beta-mercaptoethanol (500 μM) at 37 degrees Celsius with 5% CO2. Approximately 1×10⁷ cells were collected, pelleted by centrifugation, and washed 3× with 1 ml of PBS (pH 7.4) at room temperature. RNA was extracted using the RNeasy mini kit (Quiagen, Valencia, Calif.) and a cDNA library was generated using oligo dt reverse transcription. Expressed Chicken IgM constant regions 1-3 were amplified by PCR using this cDNA library as a template. The primers used to amplify chicken IgM C1-C3 contained either a PshAI (forward primer—GACCAAAGTCATCGTCTCCTCCGCCT, SEQ ID NO 5) or an SgraI (reverse primer—CGCCGGTGCCAGTGTGCTGGAATTCG, SEQ ID NO 6) restriction enzyme site over hang. The resultant PCR product was cloned into the pCR4 TOPO-TA vector as per manufacture's protocols (Invitrogen, Carlsbad Calif.) and chicken IgM containing plasmids were prepared using standard methods. Chicken IgM C1-C3 was removed from pCR4 TOPO by restriction enzyme digestion with PhsAI and SgrAI, gel purified, and ligated directionally into the PshAI and SgrAI sites of a b12 containing pDR vector (pDR/b12). Digestion of pDR/b12 removed 421 of the 1262 nucleotides constituting the b12 heavy-chain ORF, resulting in a final construct expressing a chimeric antibody heavy-chain including the variable region of b12 fused in-frame with the membrane bound form of the chicken IgM constant regions C1-C3 (SEQ ID NO 7 and SEQ ID NO 8).

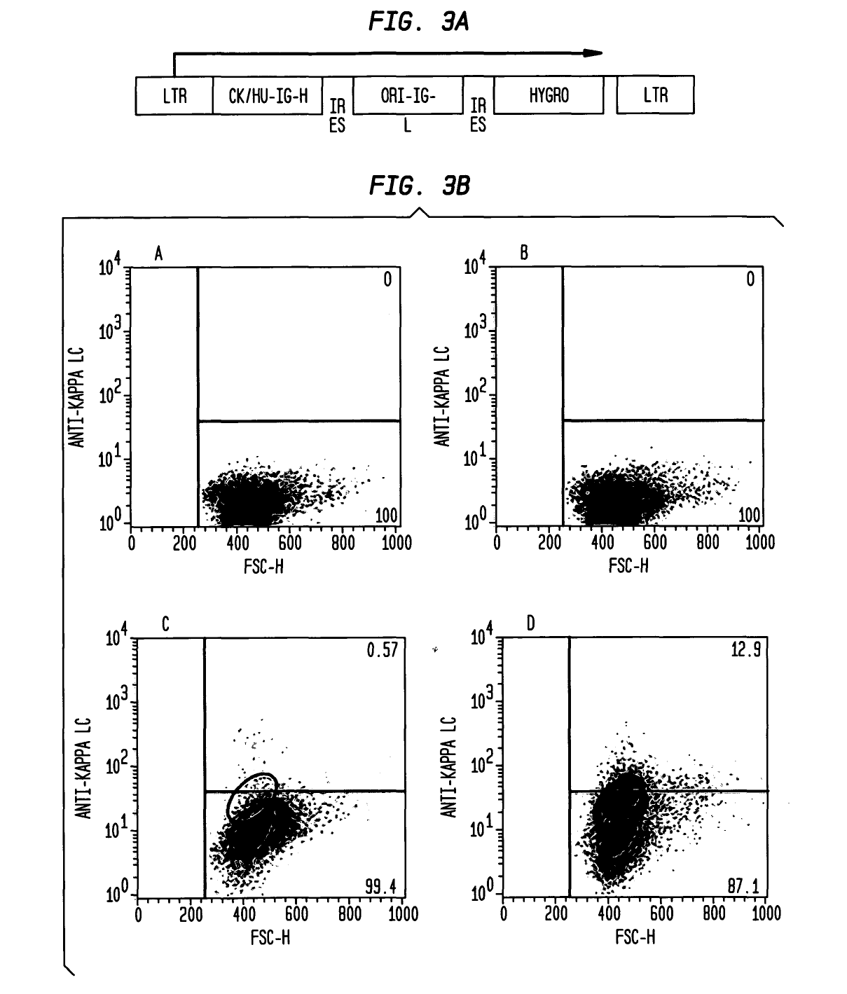

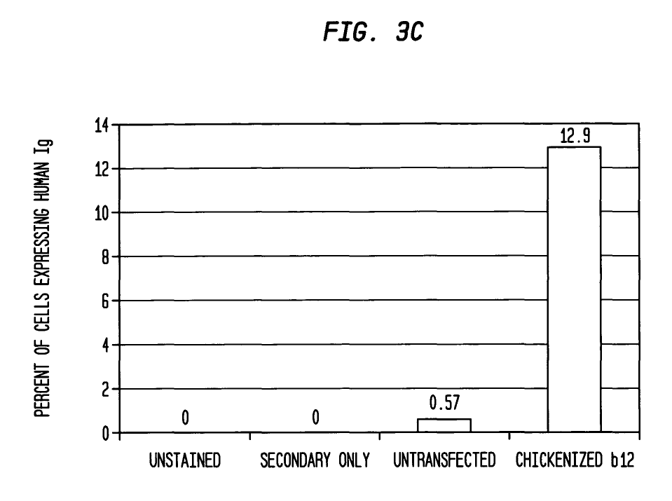

2 μg of the chicken-human hybrid b12 expression vector comprising the GFP gene as a marker was used to transfect 2 million AID^{-/-}/IgH⁻/IgL⁻ DT40 cells with lipofectamine, with a GFP expression plasmid as a positive control for transfection efficiency. Cells were then grown for 48 hrs, and transient cell-surface expression of “chickenized” b12 mIgM was analyzed and confirmed by mouse monoclonal anti-chicken IgM and anti-human kappa chain flow cytometry (FIGS. 3A and 3B; SEQ ID NO 4 and SEQ ID NO 8). This result shows that we were able to convert the human IgG1 to a chimeric IgM BCR with chicken constant regions for membrane anchoring, and interaction with proteins of the chicken BCR complex, such as Igα and Igβ.

### Example 3

### NFκB-Responsive Induction of Reporter Gene Assay

DT40 cells are transfected both with a vector driving expression of the b12 broadly neutralizing anti-HIV Env surface IgM, and with a vector reporting cRel/NFκB-responsive expression of the luciferase reporter gene (FIG. 4; SEQ ID NO 12). Doubly transfected cells are isolated by dual marker selection (GFP and RFP), and seeded in 96-well plates, as described above. In the presence and absence of CD40 ligand and T helper cytokines, cells are stimulated with the positive control, anti-IgM, to cross-link the BCR, and the max-strength signal downstream of BCR activation is generated. The luciferase signals are read on a plate-reader between 24 and 48 hours following stimulation.

Using the HIV immunogens YU2, JRFL, and DU422 as examples (SEQ ID NO 9, SEQ ID NO 10, and SEQ ID NO 11, respectively), each antibody in human peripheral mature naïve B cells is assayed at various concentrations for dose response curves. Cells are stimulated in the presence and absence of CD40 ligand and cytokines that mimic T help. Each 96-well plate contains four wells dedicated to the positive control (anti-IgM max signal concentration) and four wells dedicated to the negative control (2 with non-specific Ig at the same concentration as the anti-IgM antibody, and two with buffer alone). NFκB-responsive transcription is measured over time on a plate reader and by flow cytometry. For immunogen characterization, all data is normalized to the positive control wells, which are expressed as 100% signal. The data is analyzed as described for the Ca⁺⁺ Assays above, and the EC₅₀ value are determined, the logarithm of which is the pEC₅₀ value.

1. A method of identifying an antigen that induces BCR signaling, the method comprising expressing in a B cell immunoglobulin heavy and light chains that have neutralizing specificity, whereby the heavy chain has transmembrane and cytoplasmic domains, exposing the cell expressing the BCR with neutralizing Ab specificity to one or more antigens, and assaying for signaling downstream of BCR activation.

2. The method of claim 1, whereby the cell in which immunoglobulin heavy and light chains that have neutralizing specificity are expressed is a primary B cell.

3. The method of claim 1, whereby the cell in which immunoglobulin heavy and light chains that have neutralizing specificity are expressed is an immortalized B cell.

4. The method of claim 3, whereby the immortalizaed B cell is selected from the group consisting of DT40, Ramos and CH12 cells.

5. The method of claim 3, where the immortalized cell does not express an endogenous immunoglobulin heavy chain or an endogenough immunoglobulin light chain.

6. The method of claim 1, further comprising a means of reducing or eliminating expression of endogenous immunoglobulin expression.

7. The method of claim 6, whereby the means of reducing or eliminating expression of endogenous immunoglobulin expression is selected from the group consisting of selection, gene knock-out, and gene knock-down.

8. The method of claim 1, whereby the signaling downstream of BCR activation is a change in the concentration of second messenger in a subcellular compartment.

9. The method of claim 8, whereby the subcellular compartment is the cytoplasm.

10. The method of claim 8, whereby the second messenger is Ca⁺⁺.

11. The method of claim 1, whereby the signaling downstream of BCR activation is a biochemical change in a cellular protein

12. The method of claim 11, whereby the biochemical change is a posttranslational modification

13. The method of claim 11, whereby the biochemical change is a protein-protein or protein polynucleotide interaction.

14. The method of claim 1, whereby the signaling downstream of BCR activation is a change in the specific activity of a cellular protein.

15. The method of claim 14, whereby the cellular protein is an enzyme.

16. The method of claim 1, whereby the signaling downstream of BCR activation is a change in the subcellular localization of a cellular protein.

17. The method of claim 1, whereby the signaling downstream of BCR activation is a change in the transcription rate of gene.

18. The method of claim 17, whereby the gene is a reporter gene under the control of promoter.

19. The method of claim 18, whereby the promoter comprises one or more enhancer elements.

20. The method of claim 18, whereby the promoter comprises one or more NKkB responsive enhancer elements.
